SEQUENCE LISTING (60 sequences)

SEQ ID NO: 1 - DNA, 20 bp, Artificial sequence
  Synthetic oligonucleotide
atgagaatcaaggcggtcga

SEQ ID NO: 2 - DNA, 20 bp, Artificial sequence
  Synthetic oligonucleotide
catgagaatcaaggcggtcg

SEQ ID NO: 3 - DNA, 20 bp, Artificial sequence
  Synthetic oligonucleotide
taggcaagagtgccttgacg

SEQ ID NO: 4 - DNA, 23 bp, Artificial sequence
  Synthetic oligonucleotide
atgagaatcaaggcggtcgaggg

SEQ ID NO: 5 - DNA, 23 bp, Artificial sequence
  Synthetic oligonucleotide
catgagaatcaaggcggtcgagg

SEQ ID NO: 6 - DNA, 23 bp, Artificial sequence
  Synthetic oligonucleotide
ggcgaaggcaagccctacgaggg

SEQ ID NO: 7 - DNA, 23 bp, Artificial sequence
  Synthetic oligonucleotide
aagtgcacatccgagggcgaagg

SEQ ID NO: 8 - DNA, 23 bp, Artificial sequence
  Synthetic oligonucleotide
cacttcaagtgcacatccgaggg

SEQ ID NO: 9 - DNA, 23 bp, Artificial sequence
  Synthetic oligonucleotide
agaatcaaggcggtcgagggcgg

SEQ ID NO: 10 - DNA, 23 bp, Artificial sequence
  Synthetic oligonucleotide
atggtctgggtgccctcgtaggg

SEQ ID NO: 11 - DNA, 23 bp, Artificial sequence
  Synthetic oligonucleotide
gggcgaaggcaagccctacgagg

SEQ ID NO: 12 - DNA, 23 bp, Artificial sequence
  Synthetic oligonucleotide
gtagggcttgccttcgccctcgg

SEQ ID NO: 13 - DNA, 23 bp, Artificial sequence
  Synthetic oligonucleotide
ccacttcaagtgcacatccgagg

SEQ ID NO: 14 - DNA, 23 bp, Artificial sequence
  Synthetic oligonucleotide
catggtctgggtgccctcgtagg

SEQ ID NO: 15 - DNA, 23 bp, Artificial sequence
  Synthetic oligonucleotide
gccaggatgtcgaaggccaaggg

SEQ ID NO: 16 - DNA, 23 bp, Artificial sequence
  Synthetic oligonucleotide
ctcgaccgccttgattctcatgg

SEQ ID NO: 17 - DNA, 23 bp, Artificial sequence
  Synthetic oligonucleotide
agccaggatgtcgaaggcgaagg

SEQ ID NO: 18 - DNA, 23 bp, Artificial sequence
  Synthetic oligonucleotide
tttgctgccgtacatgaagctgg

SEQ ID NO: 19 - DNA, 39 bp, Artificial sequence
  Synthetic oligonucleotide
gccaccgctagccttgccaccatggacaagaagtactcc

SEQ ID NO: 20 - DNA, 48 bp, Artificial sequence
  Synthetic oligonucleotide
aagacttcctctgccctcagccaccttcctcttcttcttggggtcagc

SEQ ID NO: 21 - DNA, 22 bp, Artificial sequence
  Synthetic oligonucleotide
gctgagggcagaggaagtcttc

SEQ ID NO: 22 - DNA, 36 bp, Artificial sequence
  Synthetic oligonucleotide
gccaccgcggccgcttagacgttgatcctggcgctg

SEQ ID NO: 23 - DNA, 20 bp, Artificial sequence
  Synthetic oligonucleotide
accgagaccctgtaccccgc

SEQ ID NO: 24 - DNA, 20 bp, Artificial sequence
  Synthetic oligonucleotide
cagggccatgtcggctctgc

SEQ ID NO: 25 - DNA, 24 bp, Artificial sequence
  Synthetic oligonucleotide
ggaaccaattcagtcgactggatc

SEQ ID NO: 26 - DNA, 60 bp, Artificial sequence
  Synthetic oligonucleotide
tttcttggctttatatatcttgtggaaaggacgaaacaccgaggcaagagtgccttgacg

SEQ ID NO: 27 - DNA, 60 bp, Artificial sequence
  Synthetic oligonucleotide
gactagccttattttaacttgctatttctagctctaaaaccgtcaaggcactcttgcctc

SEQ ID NO: 28 - DNA, 60 bp, Artificial sequence
  Synthetic oligonucleotide
tttcttggctttatatatcttgtggaaaggacgaaacaccgtgagaatcaaggcggtcga

SEQ ID NO: 29 - DNA, 60 bp, Artificial sequence
  Synthetic oligonucleotide
gactagccttattttaacttgctatttctagctctaaaactcgaccgccttgattctcac

SEQ ID NO: 30 - DNA, 60 bp, Artificial sequence
  Synthetic oligonucleotide
tttcttggctttatatatcttgtggaaaggacgaaacaccgatgagaatcaaggcggtcg

SEQ ID NO: 31 - DNA, 60 bp, Artificial sequence
  Synthetic oligonucleotide
gactagccttattttaacttgctatttctagctctaaaaccgaccgccttgattctcatc

SEQ ID NO: 32 - DNA, 37 bp, Artificial sequence
  Synthetic oligonucleotide
gccaccccaccgagacctcagtcgactggatccggta

SEQ ID NO: 33 - DNA, 36 bp, Artificial sequence
  Synthetic oligonucleotide
gccaccggtctcggtgggatccactagtaacggccg

SEQ ID NO: 34 - DNA, 38 bp, Artificial sequence
  Synthetic oligonucleotide
gccaccgggcccatggtgagcgagctgattaaggagaa

SEQ ID NO: 35 - DNA, 38 bp, Artificial sequence
  Synthetic oligonucleotide
gccaccggccggccttatctgtgccccagtttgctagg

SEQ ID NO: 36 - DNA, 60 bp, Artificial sequence
  Synthetic oligonucleotide
gccaccgggcccatgagaatcaaggcggtcgaggggatgagcgggggcgaggagctgttc

SEQ ID NO: 37 - DNA, 38 bp, Artificial sequence
  Synthetic oligonucleotide
gccaccggccggccttagacgttgatcctggcgctggc

SEQ ID NO: 38 - DNA, 60 bp, Artificial sequence
  Synthetic oligonucleotide
gccaccgggcccatgagaatcaaggcggtcgatgggatgagcgggggcgaggagctgttc

SEQ ID NO: 39 - DNA, 60 bp, Artificial sequence
  Synthetic oligonucleotide
gccaccgggcccatgagaatcaaggctgtagatgggatgagcgggggcgaggagctgttc

SEQ ID NO: 40 - DNA, 44 bp, Artificial sequence
  Synthetic oligonucleotide
tgtggataaccgtattaccgccccgtacacgccacctcgacata

SEQ ID NO: 41 - DNA, 44 bp, Artificial sequence
  Synthetic oligonucleotide
gccaccgctagccggaaagttggtataagacaaaagtgttgtgg

SEQ ID NO: 42 - DNA, 22 bp, Artificial sequence
  Synthetic oligonucleotide
cggccgttactagtggatccgc

SEQ ID NO: 43 - DNA, 30 bp, Artificial sequence
  Synthetic oligonucleotide
ggcggtaatacggttatccacagaatcagg

SEQ ID NO: 44 - DNA, 20 bp, Artificial sequence
  Synthetic oligonucleotide
tggaaaggacgaaacaccgt

SEQ ID NO: 45 - DNA, 20 bp, Artificial sequence
  Synthetic oligonucleotide
ttttctacggggtctgacgc

SEQ ID NO: 46 - DNA, 20 bp, Artificial sequence
  Synthetic oligonucleotide
tttgctcggcaccttgtact

SEQ ID NO: 47 - DNA, 23 bp, Artificial sequence
  Synthetic oligonucleotide
atgagaatcggaatggtcgaggg

SEQ ID NO: 48 - DNA, 23 bp, Artificial sequence
  Synthetic oligonucleotide
atgagaatcggaacggtcgaggg

SEQ ID NO: 49 - DNA, 23 bp, Artificial sequence
  Synthetic oligonucleotide
atgagaatcagaacggtcgaggg

SEQ ID NO: 50 - DNA, 23 bp, Artificial sequence
  Synthetic oligonucleotide
atgagaatcagagcggtcgaggg

SEQ ID NO: 51 - DNA, 23 bp, Artificial sequence
  Synthetic oligonucleotide
atgagaatcaaagcggtcgaggg

SEQ ID NO: 52 - DNA, 23 bp, Artificial sequence
  Synthetic oligonucleotide
atgagaatcaaggctgtagatgg

SEQ ID NO: 53 - DNA, 23 bp, Artificial sequence
  Synthetic oligonucleotide
gcgagaatcaaggcggtcgaggg

SEQ ID NO: 54 - DNA, 23 bp, Artificial sequence
  Synthetic oligonucleotide
gtgagaatcaaggcggtcgaggg

SEQ ID NO: 55 - DNA, 23 bp, Artificial sequence
  Synthetic oligonucleotide
atgagaatcaaggcggtcgatgg

SEQ ID NO: 56 - DNA, 36 bp, Artificial sequence
  Synthetic oligonucleotide
  misc_feature (2)..(6): n is a, c, g, t or u
  misc_feature (29)..(36): n is a, c, g, t or u
mnnnnntgrkmaycarggyggkcsagggnnnnnnnn

SEQ ID NO: 57 - DNA, 36 bp, Artificial sequence
  Synthetic oligonucleotide
cgagggtggtcaccagggtgggccagggcacgggca

SEQ ID NO: 58 - protein, 1680 aa, Artificial sequence
  Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAEGRGSLLTCGDVEENPGPSGGPMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRGGGGSGSHGFPPEVEEQAAGTLPMSCAQESGMDRHPAACASARINV

SEQ ID NO: 59 - protein, 1634 aa, Artificial sequence
  Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAEGRGSLLTCGDVEENPGPSGGPMVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 60 - protein, 1689 aa, Artificial sequence
  Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVAEGRGSLLTCGDVEENPGPMRIKAVEGMSGGEELFAGIVPVLIELDGDVHGHKFSVRGEGEGDADYGKLEIKFICTTGKLPVPWPTLVTTLAWGIQCFARYPEHMKMNDFFKSAMPEGYIQERTIHFQDDGKYKTRGEVKFEGDTLVNRVELKGEGFKEDGNILGHKLEYSAISDNVYIMPDKANNGLEANFKIRHNIEGGGVQLADHYQTNVPLGDGPVLIPINHYLSCQSAISKDRNEARDHMVLLESFSAYCHTHGMDELYRSGLRSSHGFPPEVEEQAAGTLPMSCAQESGMDRHPAACASARINV

(19) United States
(12) Patent Application Publication
(10) Pub. No.: US 2018/0187172 A1
(43) Pub. Date: Jul. 5, 2018

(54) REGULATED GENE EXPRESSION FROM VIRAL VECTORS

(71) Applicant: Board of Regents, the University of Texas System

(72) Inventors: Leonidas BLERIS, Allen, TX (US); Richard Taplin MOORE, Frisco, TX (US)

(21) Appl. No.: 15/323,237

(22) PCT Filed: Jun. 30, 2015
(86) PCT No.: PCT/US2015/038497

§ 371 Date: Dec. 30, 2016

(51) Int. Cl. C12N 9/22; C12N 15/11; C12N 15/86

(52) U.S. Cl. CPC C12N 9/22; C12N 2310/20; C12N 15/86; C12N 15/11

(57) ABSTRACT

The present disclosure relates to vectors for the controlled expression transgenes and methods of use therefor.

This application claims benefit of priority to U.S. Provisional Application Ser. No. 62/019,605, filed Jul. 1, 2014, the entire contents of which are hereby incorporated by reference.

The invention was made with government support under grant nos. GM098984, GM096271 and CA17001801 awarded by the National Institutes of Health (NHLBI), and grant no. CBNET-1105524 awarded by the U.S. National Science Foundation (NSF). The government owns certain rights in the invention.

### BACKGROUND OF THE INVENTION

### 1. Field of the Invention

The present invention relates generally to the fields of molecular biology and virology. More particularly, it concerns regulation of transgene expression from non-viral and viral vectors. Specifically, the invention relates to the use of regulatory machinery to control the expression of heterologous genes delivered into mammalian cells.

### 2. Description of Related Art

The ability to deliver genes into cells and provided for regulated expression remains an important research tool. However, commercially available vector-based systems (typically lipid-based delivery) result in transient expression of the gene of interest for several days, and often result in random integrations in the genome of the transfected cells. For example, herpes simplex virus (HSV)/viral expression systems, currently in phase III clinical trials, allow the expression of larger genomic constructs than Adenoviral or Lentivirus systems. Commercially available HSV expression vectors have the capability to target specific tissue types and can also target all mammalian systems. After the HSV vector is transduced into the target cell and is transported to the nucleus, it does not integrate into the host genome but rather is expressed from the episomic genome. In the case of short term HSV vectors, expression of the transgene occurs in a few hours after injection and lasts up to 10 days. With long term HSV vectors, expression has been found to last for weeks in neighboring neural tissue but does disappear at the site of infection after a few days. Thus, finding approaches to regulate expression of HSV and other vectors is of great importance.

### SUMMARY OF THE INVENTION

Thus, in accordance with the present invention, there is provided a vector comprising (a) a heterologous nucleotide sequence under the control of a promoter active in eukaryotic cells; (b) a coding region for Cas9 protein under the control of a promoter active in eukaryotic cells; (c) a coding region for a gRNA under the control of a promoter active in eukaryotic cells wherein said gRNA targets a sequence in said delivery vector encoding said nucleotide of interest; and (d) sequences required for expression of said heterologous nucleotide sequence, Cas9 coding region and said gRNA coding region.

The vector may be a DNA plasmid delivered via non-viral methods or via viral methods. The viral vector may be a retroviral vector, a herpesviral vector, an adenoviral vector, an adeno-associated viral vector or a poxviral vector. The heterologous nucleotide sequence may encode a therapeutic gene product. The heterologous nucleotide sequence may encode a non-therapeutic detectable and/or selectable gene product. The promoters may be active in mammalian cells. At least one of said promoters may be a viral promoter. One promoter may direct expression of both said heterologous gene sequence and said Cas9 coding region. The heterologous gene sequence may further comprise a coding sequence for a protein degradation tag.

In another embodiment, there is provided a method of expressing a protein of interest in a target cell comprising (a) transferring a vector into said cell, said vector comprising (i) a nucleotide sequence encoding said protein of interest under the control of a promoter active in eukaryotic cells; (ii) a coding region for Cas9 protein under the control of a promoter active in eukaryotic cells; (iii) a coding region for a gRNA under the control of a promoter active in eukaryotic cells wherein said gRNA targets a sequence in said delivery vector encoding said nucleotide of interest; and (iv) sequences required for expression of said nucleotide sequence encoding said protein of interest, Cas9 coding region and said gRNA coding region, and (b) culturing said cell under conditions supporting expression of said protein of interest.

The viral vector may be a retroviral vector, a herpesviral vector, an adenoviral vector, an adeno-associated viral vector or a poxviral vector. The protein of interest may be a therapeutic gene product or a non-therapeutic gene product. The promoters may be active in mammalian cells. One of said promoters may be a viral promoter. One promoter may direct expression of both said gene sequence encoding said protein of interest and said Cas9 coding region. The nucleotide sequence encoding said protein of interest may further comprise a coding sequence for a protein degradation tag. The Cas9 coding region may further comprise a coding sequence for a protein degradation tag.

The cell may be an animal cell or a plant cell, such as one located in a living animal or plant. The method may further comprise detecting expression of said protein of interest, and may be underexpressed or not expressed in said cell as relative to the normal level in a comparable cell. The protein of interest may be endogenous to said cell or not endogenous to said cell. The cell may be a cancer cell or a neuronal cell.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-I. CRISPR-based delivery vehicle. (FIG. 1A) The gRNA-Cas9 complex targets template DNA. (FIG. 1B) The concept of the inventors' self-cleaving plasmid for pulsed delivery. (FIG. 1C) Representation of the inventors' single plasmid expression system with RNA Polymerase III transcript U6-gRNA combined with RNA Polymerase II Transcript CMV-Cas9-2A-mKate2-PEST. (gRNA Target=SEQ ID NO: 4) (FIG. 1D) Fluorescence microscopy indicating pulsed behavior of single plasmid construct with T1 gRNA and off-target gRNA control showing sustained mKate2 fluorescence in HEK293 cells. (FIG. 1E) Cropped images following the mKate2 dynamics in single cells. (FIG. 1F) Single cell tracks showing mean fluorescence (FIGS. 1G-I) In silico experiments of the pulse trajectory generated in MATLAB. (FIG. 1G) Titrations of the pulse plasmid create greater amplitude without a significant impact in residence time. (FIG. 1H) Increasing protein stability results in higher amplitude and longer pulse duration. (FIG. 1I) Decreasing the gRNA-Cas9 complex targeting affinity increases pulse amplitude and duration.

FIGS. 2A-J. Properties of delivery using modified CRISPR-based vehicle. (FIG. 2A) Removal of the PEST domain from the single plasmid creates a pulse of greater amplitude and residence time at equal mass transfection. Time points are shown as average mKate2 intensity of triplicate flow cytometry measurements. (FIG. 2B) Representative flow cytometry plots showing smoothed density plots of mKate2 intensity versus SSC. Representative microscopy at each time point overlaid at top right corner. (FIG. 2C) Construction of mutant targets to test modification to Cas9-gRNA affinity for the plasmid. (T1 target=SEQ ID NO: 4; Single mutant target=SEQ ID NO: 55; Triple mutant target=SEQ ID NO: 52) (FIG. 2D) Single mutant at the 5′ NGG 3′ shows similar behavior to T1 control while triple mutant has higher amplitude and residence time. Mean of triplicate flow cytometry measurements at four time points shown. (FIG. 2E) Representative microscopy of original T1 target with single and triple mutant at 24 and 48 hours expressing Tag-CFP PEST with phase images above and below. (FIG. 2F) Increasing transfection mass in overlaid flow cytometry histograms. (FIG. 2G) Mean mKate2 fluorescence intensity from two independent experiments at 24 and 48 hours indicate control of amplitude by mass titration. (FIG. 2H) Titrations of Doxycycline show change in amplitude for fixed mass transfections at 24, 48 and 72 hours. (FIG. 2I) The triple mutant and the system without the PEST domain yield the same maximum output levels and a similar trajectory. The inventors provide representative microscopy snapshots at 24 hours. (FIG. 2J) The original plasmid and the vector lacking the PEST domain. The output lacking the PEST domain remains stable in the cellular milieu approximately twice as long as the output carrying the fast degradation domain. We provide representative microscopy snapshots at 24 hours. Error bars correspond to the standard deviation between triplicate experiments.

FIG. 3. Triplicate flow cytometry time lapse of 100 ng of each plasmid. Control contains the off-target gRNA while T1 and T2 contain the gRNAs targeting the mKate2 ORF. Error bars show standard deviation of the calculated means from triplicates.

FIG. 4. Nucleotides 166 to 182 of the ORF of Tag-CFP are shown. Alignment with the T1 gRNA indicates a protospacer with 9 mismatches. (Consensus=SEQ ID NO: 56; Tag-CFP=SEQ ID NO: 57; ORF1=SEQ ID NO: 4)

FIG. 5. Triplicate flow cytometry time lapse of 100 ng of each plasmid. Control contains the off-target gRNA complete plasmid. T1 and T1 No PEST have a gRNA targeting the mKate2 ORF. The No PEST version of the T1 gRNA complete plasmid lacks a PEST domain on the mKate2 reporter. Error bars represent standard deviation of the means.

FIG. 6. Representative FACS analysis. Tag-CFP au intensity versus SSC for the original T1 Target, the single mutant and triple mutant.

FIG. 7. Representative FACS analysis. mKate2 au intensity versus SSC for the titrations of 25 ng, 100 ng and 250 ng of the original pulse U6-gRNATA-CMV-Cas9-2A-mKate2-PEST.

FIG. 8. Duplicates of 100 ng of each plasmid. Control is original U6-gRNA-T1_CMV-Cas9-2A-mKate2-PEST pulse system recorded with a voltage of 375. All Dox labeled constructs are the U6-gRNA-T1_TRE3G-Cas9-2A-mKate2-PEST under induction of Doxycycline. Error bars show standard deviation of the calculated means.

FIG. 9. Representative FACS analysis. mKate2 au intensity versus SSC for the DOX titrations of 0.0 μg, 0.1 μg, 0.25 μg, and 1.0 μg; construct is U6-gRNAT1-TRE3 G-Cas9-2A-mKate2-PEST.

FIG. 10. Reporter expression for Triple Mutant (Tag-CFP) and Pulse without PEST (mKate2) with titrations observed at 24 hours. Singlet tests indicated that a level of 10 ng of No PEST (U6-gRNAT1-CMV-Cas9-2A-mKate2-PEST) nearly matches the fluorescence (au) of 100 ng Triple Mutant (U6-gRNAT1-CMV-Cas9-2A-TagCFP-PEST).

FIG. 11. Sequences of constructs.

FIG. 12. Library of gRNA targets measurements. The output of the CRISPR-based delivery system for a different gRNA target mutants measured at 24 and 48 hours.

FIG. 13. Library of gRNA targets measurements. The residence time of the delivered fluorescent protein for selected mutants. Five representative mutants and their corresponding sequences are included in the legend. (Mutant 1=SEQ ID NO: 54; Mutant 3=SEQ ID NO: 52; Mutant 4=SEQ ID NO: 51; Mutant 6=SEQ ID NO: 49; Mutant 8=SEQ ID NO: 48)

### DETAILED DESCRIPTION OF ILLUSTRATIVE EMBODIMENTS

The inventors have developed a new method to control amplitude and duration of gene delivery in vector-based systems. The mechanism relies on the clustered regularly interspaced short palindromic repeats (CRISPR) protein Cas9. The CRISPR protein Cas9, guided by a short RNA sequence (gRNA), cleaves the delivery vector and thus inhibits its own production but also the production of a coexpressed gene of interest. They have simulation and experimental results that show that control of the amplitude and duration of gene delivery. The mechanism relies on specific combinations of: promoters, genes, gRNAs, gRNA recognition sites, and protein degradation tags. These and other aspects of the invention are described in detail below.

### I. CRISPR/CAS SYSTEM

A. CRISPR/CAS

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

Repeats were first described in 1987 for the bacterium Escherichia coli. In 2000, similar clustered repeats were identified in additional bacteria and archaea and were termed Short Regularly Spaced Repeats (SRSR). SRSR were renamed CRISPR in 2002. A set of genes, some encoding putative nuclease or helicase proteins, were found to be associated with CRISPR repeats (the cas, or CRISPR-associated genes).

In 2005, three independent researchers showed that CRISPR spacers showed homology to several phage DNA and extrachromosomal DNA such as plasmids. This was an indication that the CRISPR/cas system could have a role in adaptive immunity in bacteria. Koonin and colleagues proposed that spacers serve as a template for RNA molecules, analogously to eukaryotic cells that use a system called RNA interference.

In 2007 Barrangou, Horvath (food industry scientists at Danisco) and others showed that they could alter the resistance of Streptococcus thermophilus to phage attack with spacer DNA. Doudna and Charpentier had independently been exploring CRISPR-associated proteins to learn how bacteria deploy spacers in their immune defenses. They jointly studied a simpler CRISPR system that relies on a protein called Cas9. They found that bacteria respond to an invading phage by transcribing spacers and palindromic DNA into a long RNA molecule that the cell then uses tracrRNA and Cas9 to cut it into pieces called crRNAs.

CRISPR was first shown to work as a genome engineering/editing tool in human cell culture by 2012 It has since been used in a wide range of organisms including bakers yeast (S. cerevisiae), zebra fish, nematodes (C. elegans), plants, mice, and several other organisms. Additionally CRISPR has been modified to make programmable transcription factors that allow scientists to target and activate or silence specific genes. Libraries of tens of thousands of guide RNAs are now available.

The first evidence that CRISPR can reverse disease symptoms in living animals was demonstrated in March 2014, when MIT researchers cured mice of a rare liver disorder. Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2014/0068797, which is incorporated by reference in its entirety.

B. Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek combined tracrRNA and spacer RNA into a “single-guide RNA” molecule that, mixed with Cas9, could find and cut the correct DNA targets. Jinek et al proposed that such synthetic guide RNAs might be able to be used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence.

C. gRNA

As an RNA guided protein, Cas9 requires a short RNA to direct the recognition of DNA targets (Mali et al., 2013a). Though Cas9 preferentially interrogates DNA sequences containing a PAM sequence NGG it can bind here without a protospacer target. However, the Cas9-gRNA complex requires a close match to the gRNA to create a double strand break (Cho et al., 2013; Hsu et al., 2013). CRISPR sequences in bacteria are expressed in multiple RNAs and then processed to create guide strands for RNA (Bikard et al., 2013). Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6 (Mali et al., 2013a, b). Synthetic gRNAs are slightly over 100 bp at the minimum length and contain a portion which is targets the 20 protospacer nucleotides immediately preceding the PAM sequence NGG; gRNAs do not contain a PAM sequence.

### II. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE A
Promoter and/or Enhancer

| Promoter/Enhancer | References |
|---|---|
| Immunoglobulin Heavy | Banerji et al., 1983; Gilles et al., 1983; |
| Chain | Grosschedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin Light | Queen and Baltimore, 1983; Picard et al., 1984 |
| Chain | |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase | Jaynes et al., 1988; Horlick et al., 1989; |
| (MCK) | Johnson et al., 1989 |
| Prealbumin | Costa et al., 1988 |
| (Transthyretin) | |
| Elastase I | Omitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tranche et al., 1989, 1990 |
| α-Fetoprotein | Godbout et al., 1988; Campere and Tilghman |
| | et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins | |
| (GRP94 and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch and |
| | Berg, 1986; Wang et al., 1986; Ondek et al., |
| | 1987; Kuhl et al., 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander and Haseltine, 1987; Thiesen et al., |
| | 1988; Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla and Siddiqui et al., 1986; Jameel and |
| | Siddiqui, 1986; Shaul and Ben-Levy, 1987; |
| | Spandau et al., 1988; Vannice et al., 1988 |
| Human | Muesing et al., 1987; Hauber et al., 1988; |
| Immunodeficiency | Jakobovits et al., 1988; Feng et al., 1988; |
| Virus | Takebe et al., 1988; Rosen et al., 1988; |
| | Berkhout et al., 1989; Laspia et al., 1989; |
| | Sharp et al., 1989; Braddock et al., 1989 |
| Cytomegalovirus (CMV) | Weber et al., 1984; Boshart et al., 1985; |
| | Foecking et al., 1986 |
| Gibbon Ape Leukemia | Holbrook et al., 1987; Quinn et al., 1989 |
| Virus | |

TABLE B
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV | Glucocorticoids | Huang et al., 1981; Lee |
| (mouse mammary | | et al., 1981; Majors et al., |
| tumor virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, 1990a, |
| | Antigen | 1990b |
| Proliferin | Phorbol Ester-TPA | Mordacq and Linzer, 1989 |
| Tumor Necrosis Factor | PMA | Hensel et al., 1989 |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al., 1994; Kelly et al., 1995), the α-actin promoter (Moss et al., 1996), the troponin 1 promoter (Bhaysar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (LaPointe et al., 1996) and the αB-crystallin/small heat shock protein promoter (Gopal-Srivastava, 1995), α-myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. 2A Peptide

The inventor utilizes the 2A-like self-cleaving domain from the insect virus Thosea asigna (TaV 2A peptide) (Chang et al., 2009). These 2A-like domains have been shown to function across Eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems have shown greater than 99% cleavage activity (Donnelly et al., 2001).

C. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EP 0273085).

### III. GENES OF INTEREST

Virtually any gene encoding sequence of interest may be used in the vectors and methods of the present application. While there is particular relevance to the provision of genes of interest that benefit from a tightly regulated window of expression, there is no limitation of this disclosure to such genes.

One type of genes that may be employed is an anti-cancer gene. For example, genes like apoptin, brevinin-2R, adenovirus E4orf4, Mda-7 (also known as IL-24), Noxa (a BH3-only protein of the Bcl-2 family), Parvovirus-H1 NS1, ORCTL3 (cation transporter), Par-4 and TRAIL (TNF related apoptosis-inducing ligand) are just a few. Other anticancer proteins may be called tumor suppressors, and include genes encoding for BRCA1 and BRCA2, p16, p53, p73, PTEN, CDKN1B, Cyclin-dependent kinase inhibitor 1C, Mir-145, SDHD, DLD/NP1, secreted frizzled related protein 1, TCF21, TIG1, and von Hippel-Lindau tumor suppressor.

Another cancer-related embodiment is th use of tumor antigens. Tumor cells often express specific, tumor-associated antigens, and these “TAAs” can either be associated with class I MHC molecules and recognized by tumor-specific CD8+ cells, or associated with class II MHC molecules and recognized by CD4+ cells. Consequently, these peptides can be injected into cancer patients to induce a systemic immune response that may result in the destruction of the cancer cells.

Other genes of interest include Transcription Activator-Like Effectors (TALES), microRNAs and microRNA inhibitors (antagomirs). MicroRNAs (miRNAs) are 15-22 nucleotide non-coding RNAs that play critical roles in a multitude of biological processes including cell proliferation, differentiation, survival and motility. On average, a given miRNA can regulate several hundred transcripts, and thus often viewed as master regulators of the human genome. Many miRNAs have been demonstrated to associate with cancer. Antagomirs are designed to interfere with miRNA function by hybridizing to the miRNA target.

### IV. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Recombinant DNA Constructs.

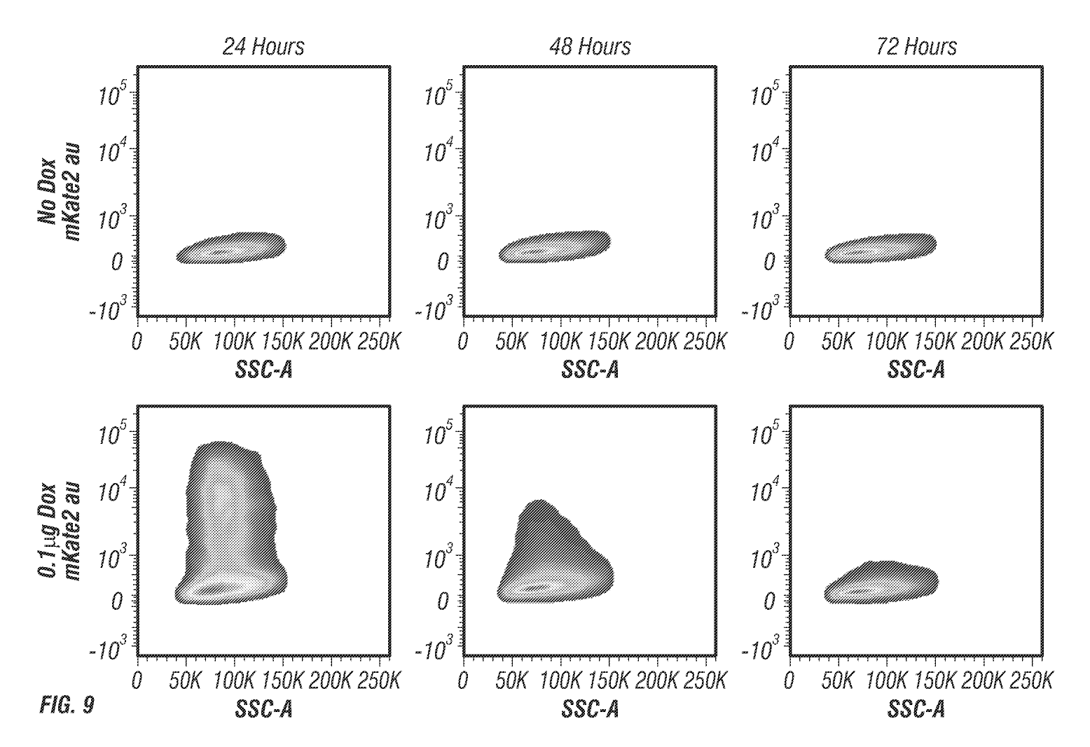

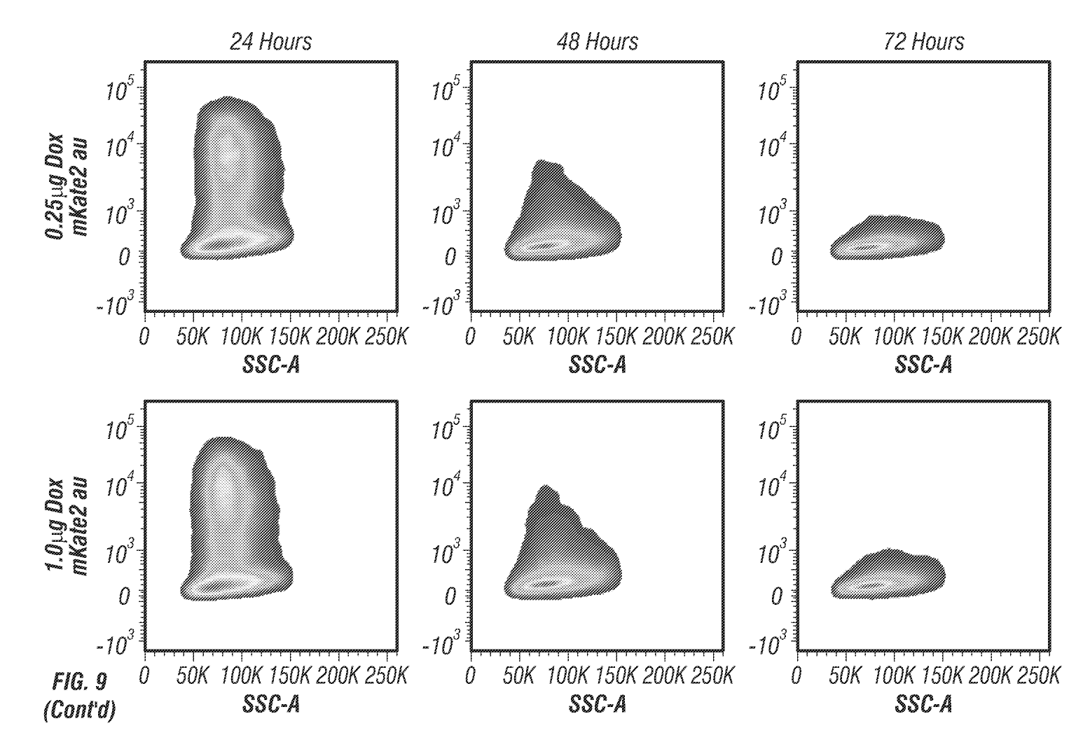

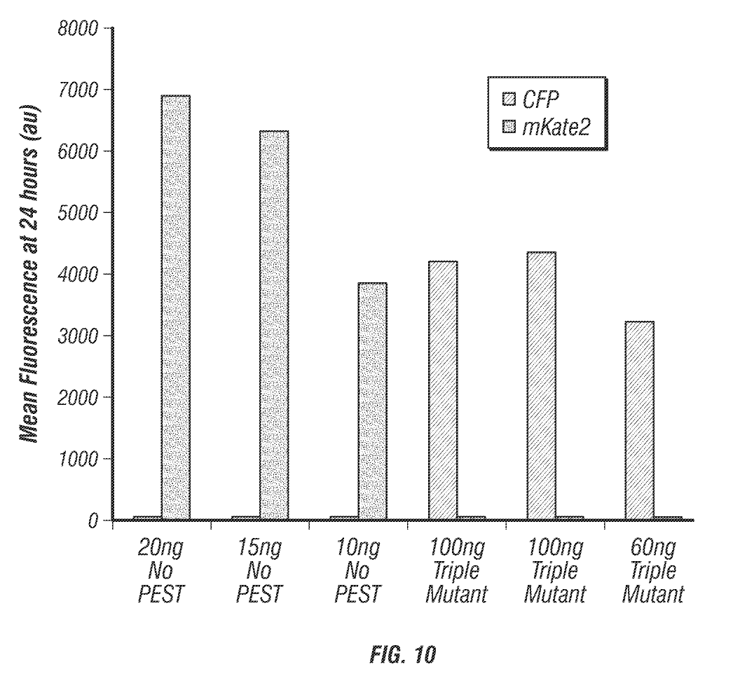

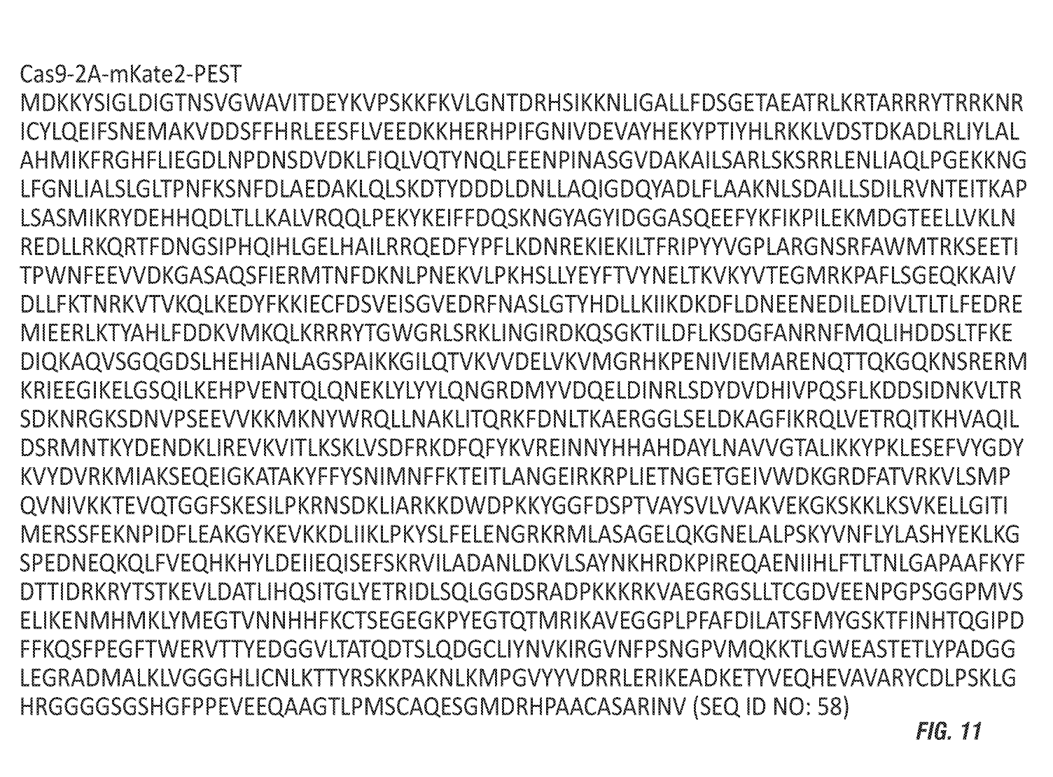

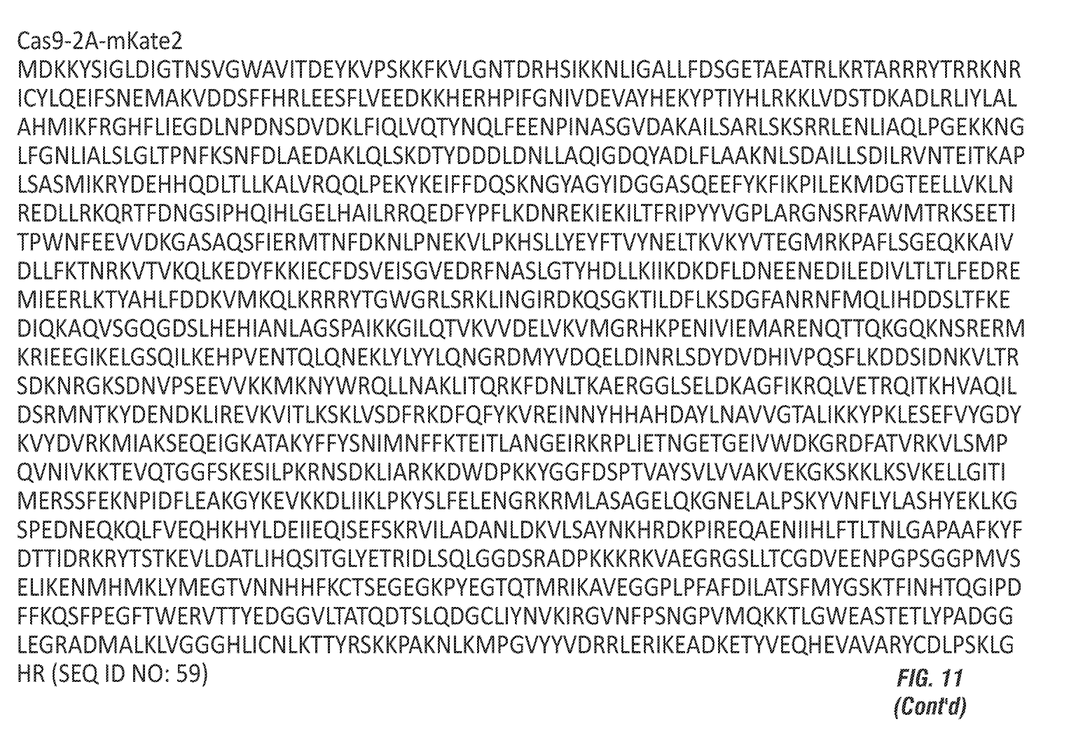

The human codon optimized Cas9 containing nuclear localization signals and an empty gRNA backbone were obtained from Addgene¹⁰. Q5 Polymerase (NEB) was utilized for all PCR product amplifications according to the manufacturer's protocol. Oligos were ordered form Sigma and are listed in Table 5. The plasmids were constructed utilizing PCR amplification, restriction digest and ligation with T4 ligase (NEB). Gel purification and PCR purification were performed with QIAquick kits (Qiagen). All plasmids over 9kb were transformed in XL10 Gold Ultracompetent Cells (Agilent) and those below 9kb were transformed in NEB 5-alpha High Efficiency E. Coli. Colonies were screened by PCR amplification using Taq (NEB) and verified by Sanger sequencing (Genewiz). DNA isolation was performed with the QIAprep miniprep kit (Qiagen). Translated open reading frames appear in FIG. 9.

Cell Culture and Transient Transfection.

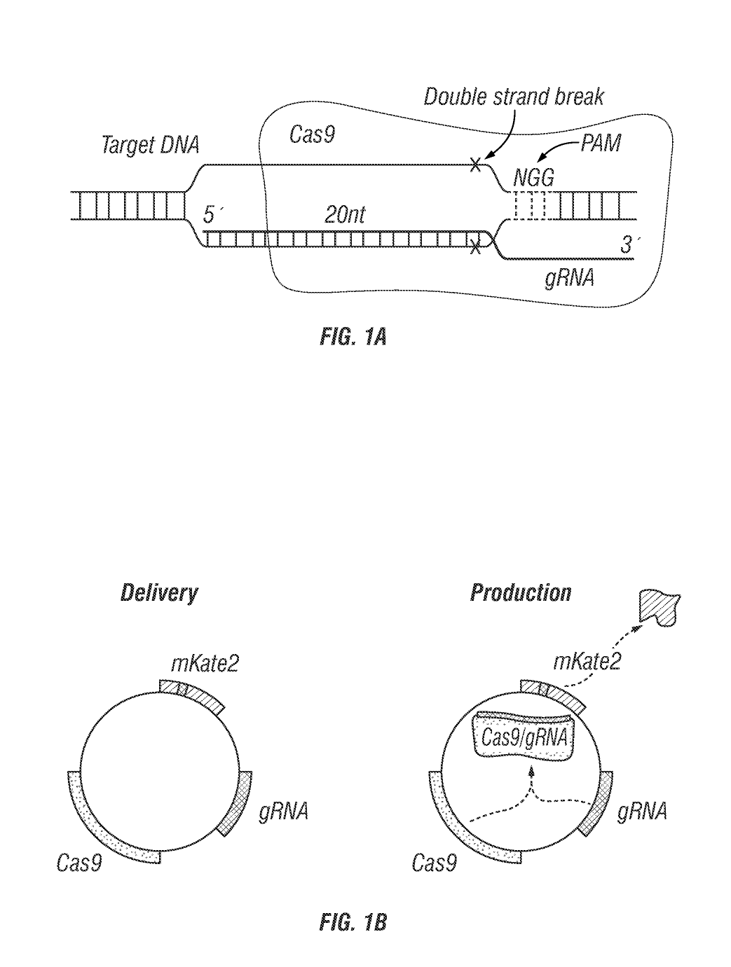

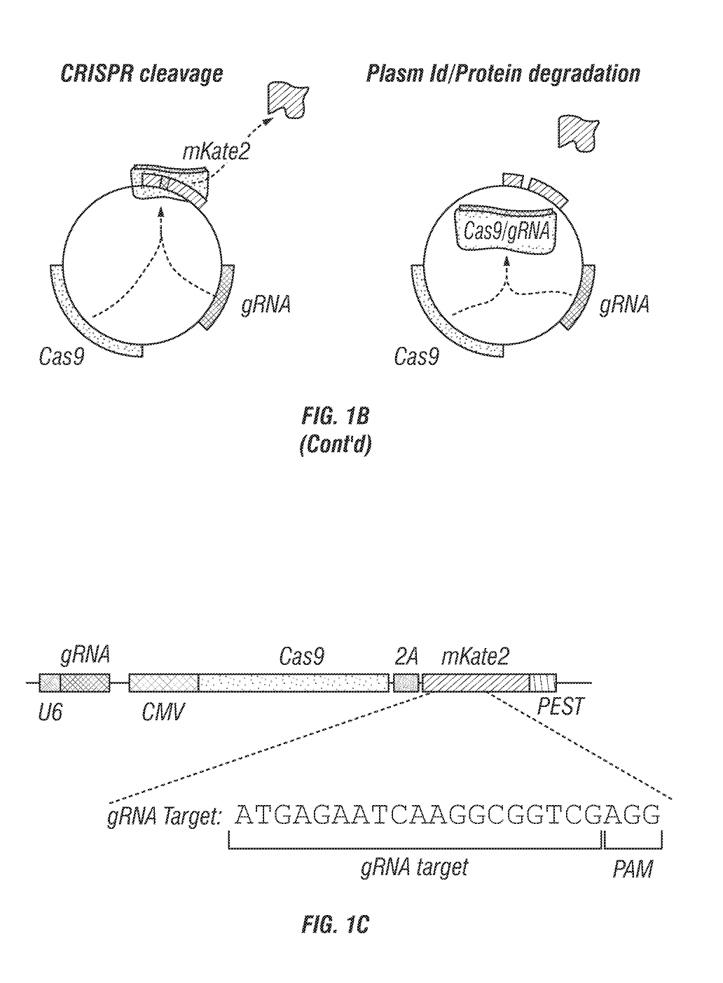

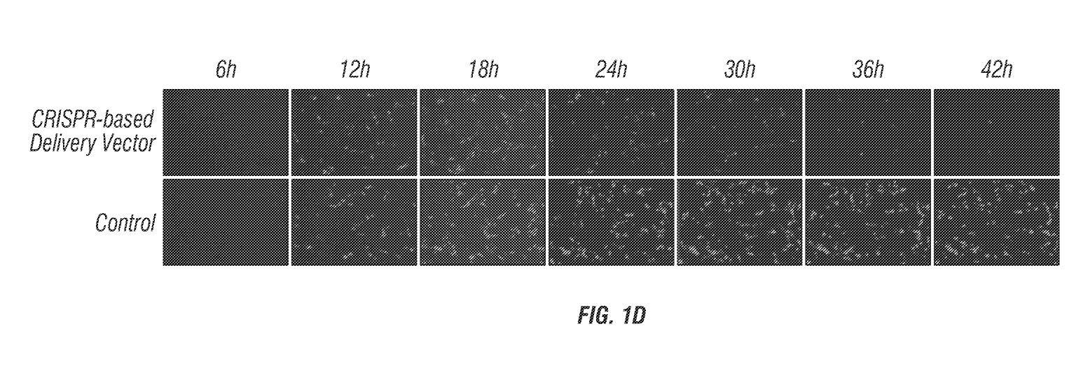

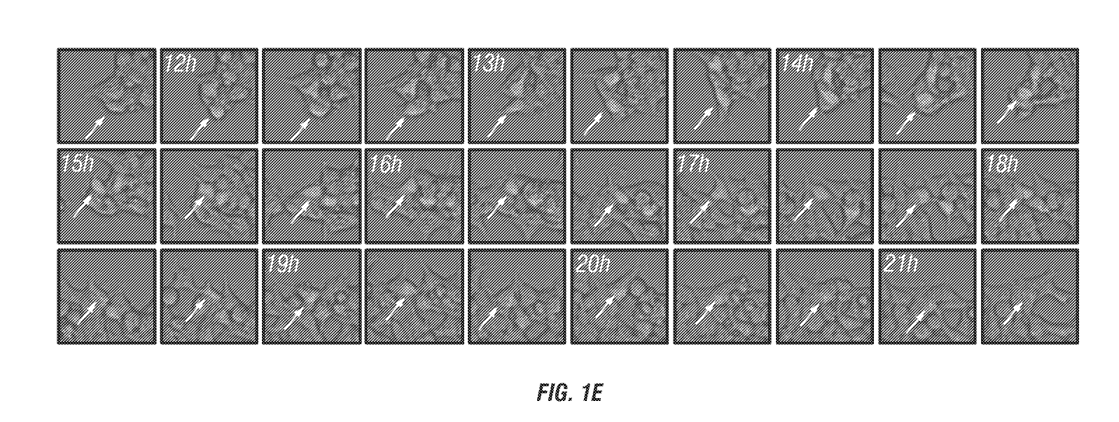

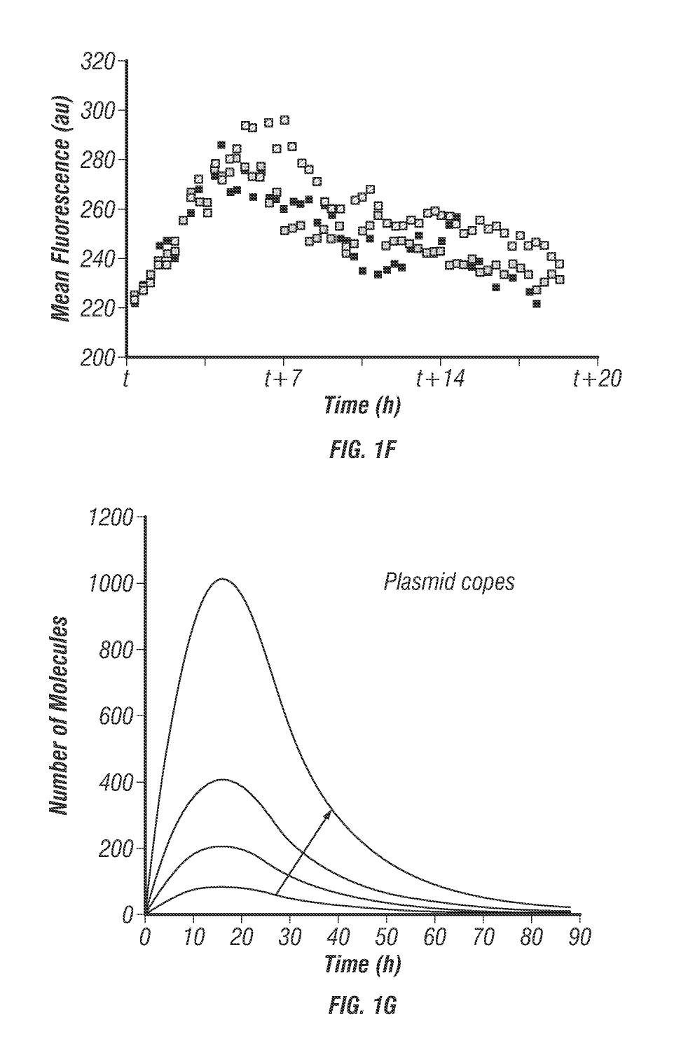

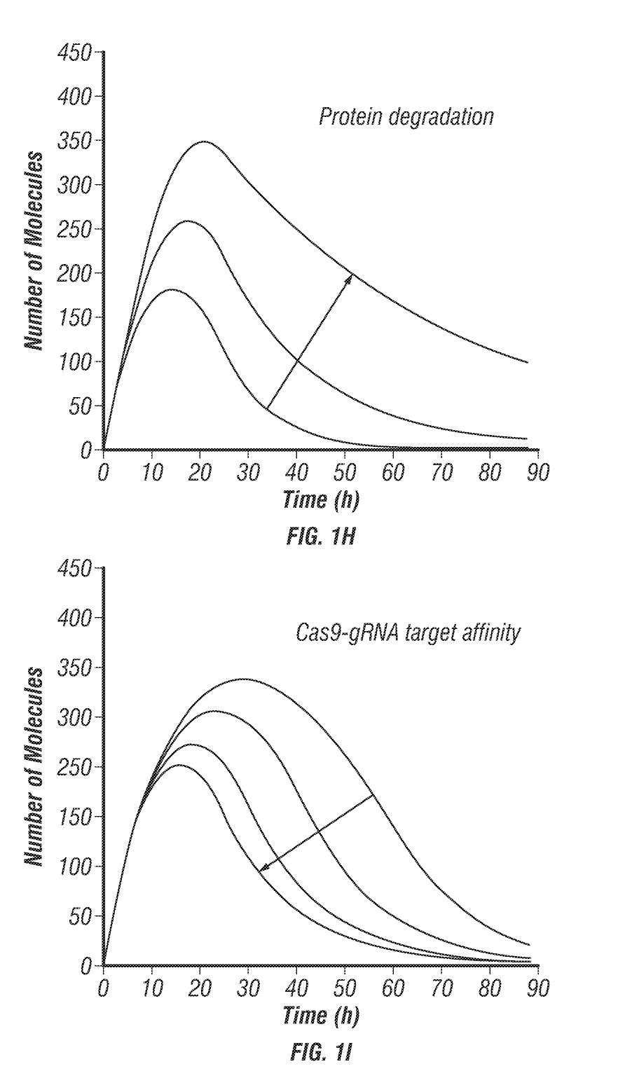

HEK293 and Tet-On HEK293 were maintained in DMEM (ThermoFisher-Life) supplemented with 9.0% FBS (Atlanta Biologicals), 0.9% MEM NEAA 100× (ThermoFisher-Gibco) and 0.4% Pen Strep (ThermoFisher-Gibco). Cells were incubated at 37° C. at 5% CO₂ and 95% humidity. All transfections were performed with JetPRIME (Polyplus) in 12-well plates (Greiner Bio One) at a plating density of 250,000 cells 24 hours before transfection according to the manufacturer's protocol. Each well received 100 ng of plasmid unless otherwise noted and 1.75 μl of JetPRIME was used per well. Cotransfection DNA was added to create a total mass of 500 ng per well with the exception of the experiment shown in FIG. 1A where a total mass of 750 ng was used. For experiments in Tet-On HEK293 cells Doxycycline was added immediately following transfection in induced wells.

Microscopy.

A Hamamatsu camera attached to the Olympus IX81 microscope with a 10× objective was used to capture images with the following filters (Chroma): ET560/40x (excitation) and ET630/75m (emission) for mKate2 and ET436/20x (excitation) and ET480/40m (emission) for Tag-CFP. The phase images were captured at 10 ms exposure and Tag-CFP at 150 ms exposure. The exposure for mKate2 was 15 ms, except as noted in selected figures. For each experiment image normalization and processing were consistent. All raw images were captured with SlideBook 5.0 (3i) and time-lapse images were captured at 20 minute intervals.

Single Cell Analysis.

Multipoint 16 bit intensity TIFF files were exported from SlideBook and processed with ImageJ. The TIFF files were exported into individual images and pre-processed with the software package Circadian Gene Expression (CGE). To quantify fluorescence, the center of fluorescence at individual cells was marked for each frame of the pulse. CGE then generated a mean intensity value for tracked cells over each frame and individual tracks were combined in Excel for analysis of amplitude and residence time.

Flow Cytometry.

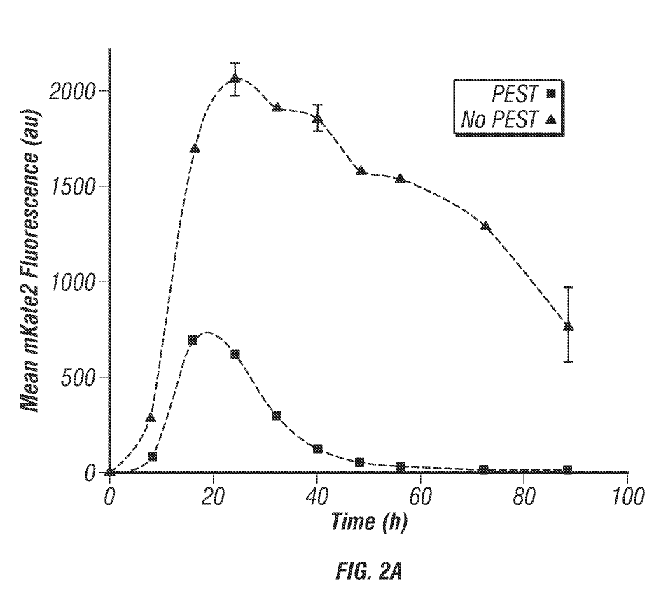

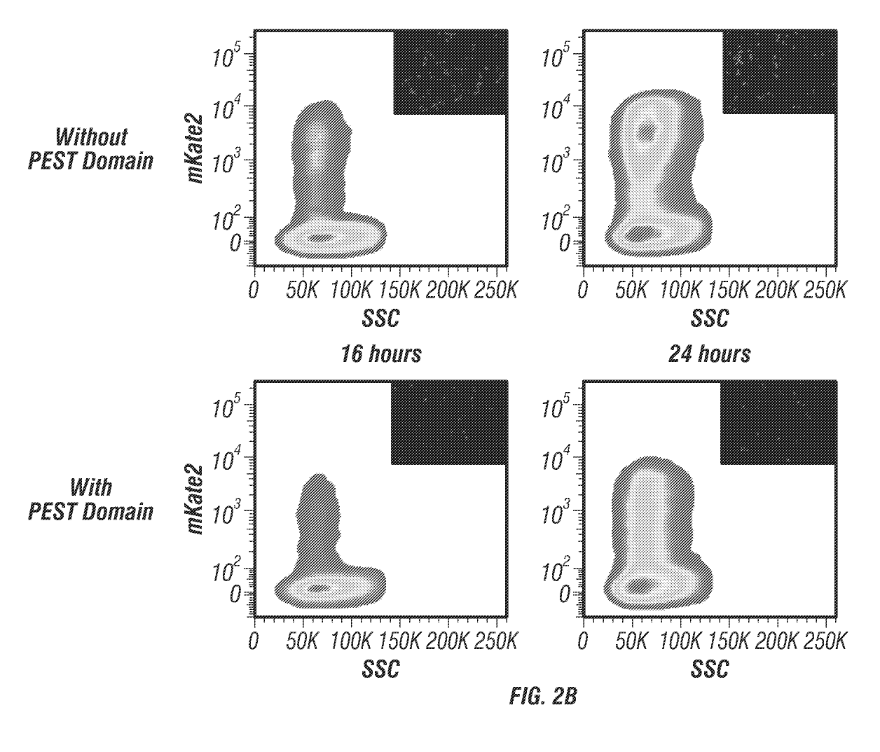

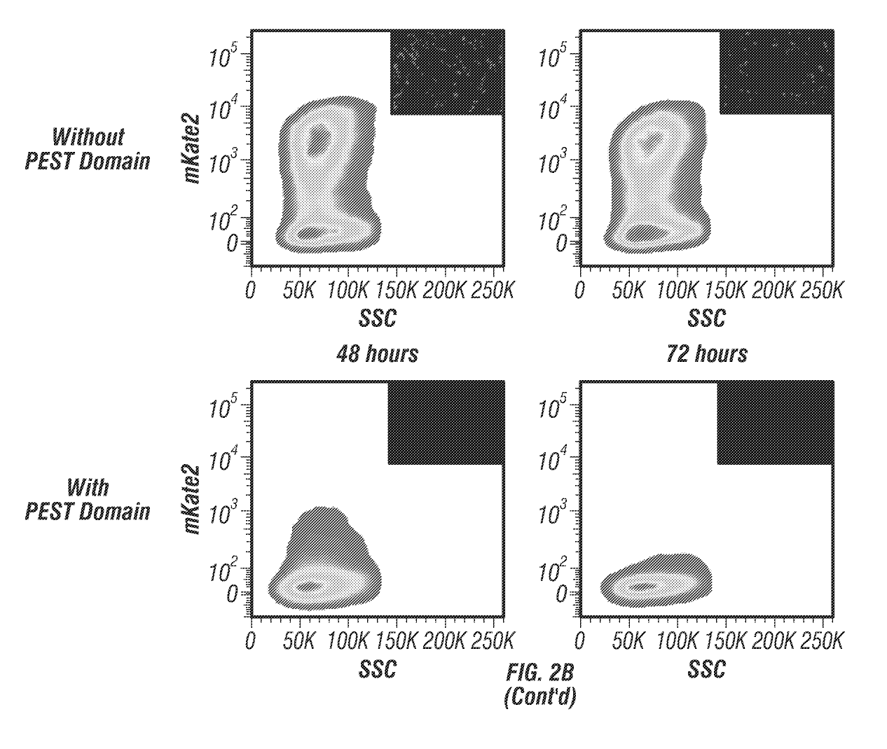

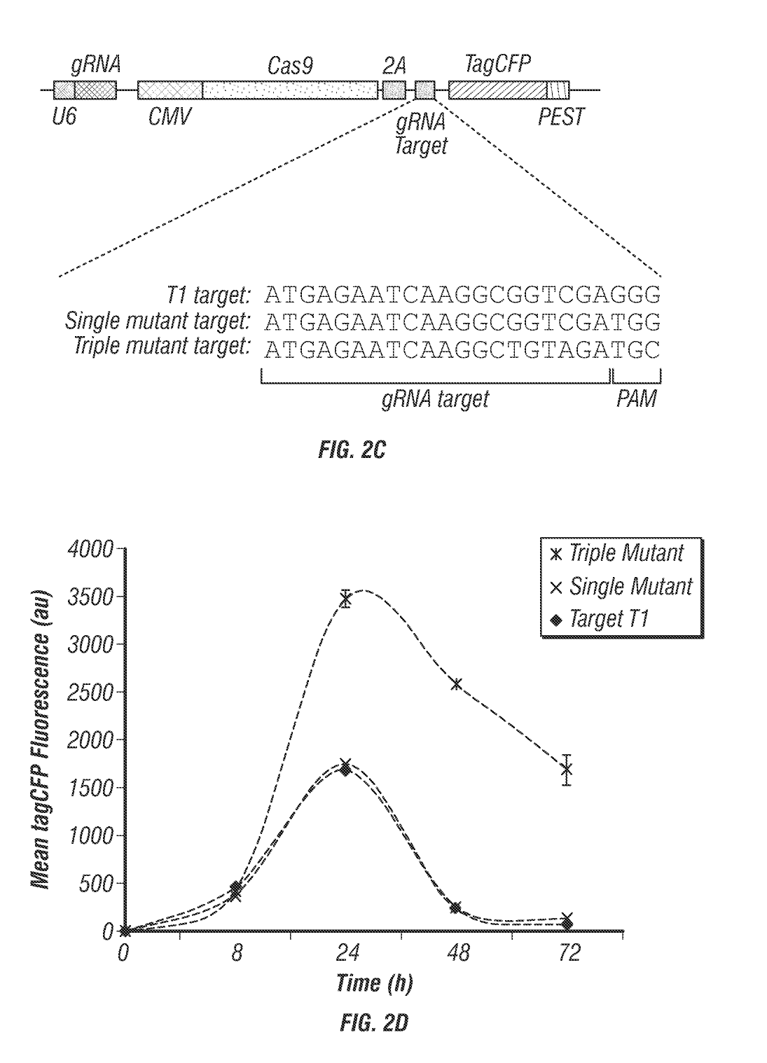

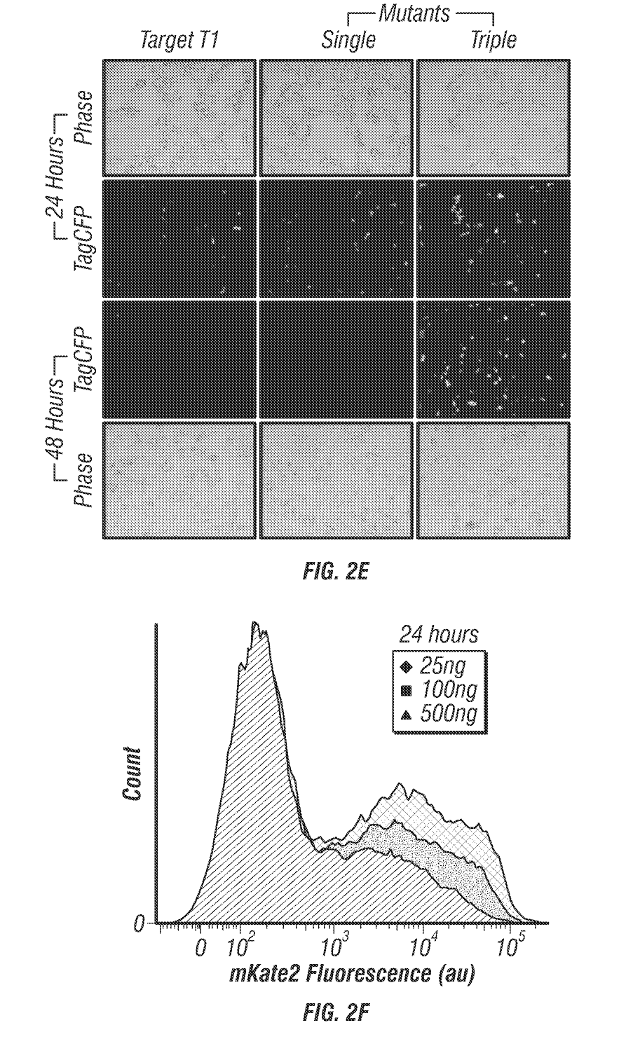

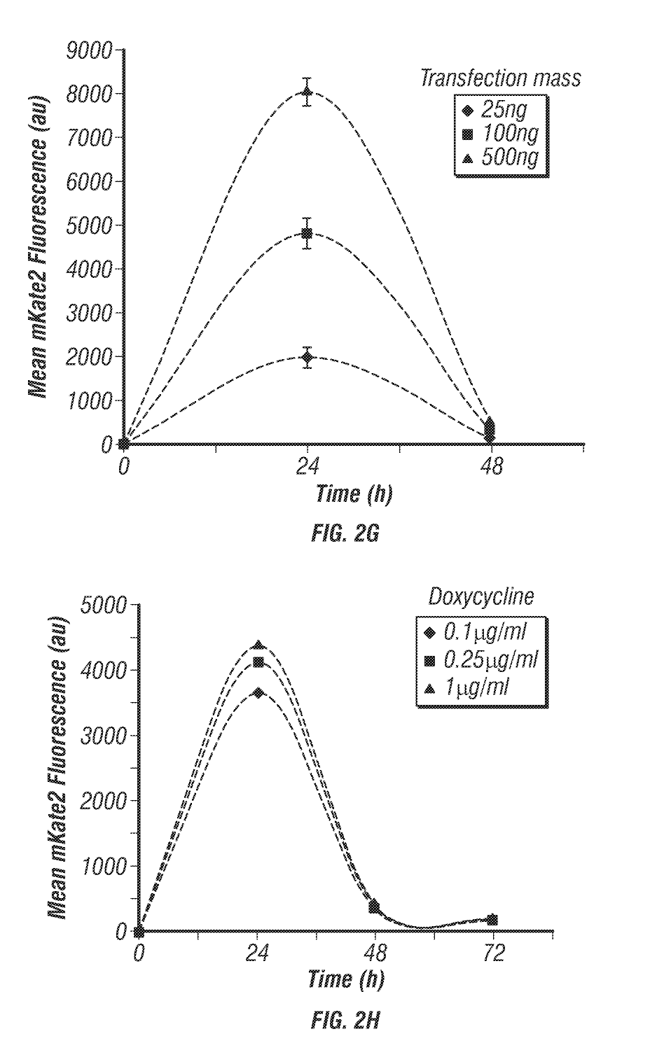

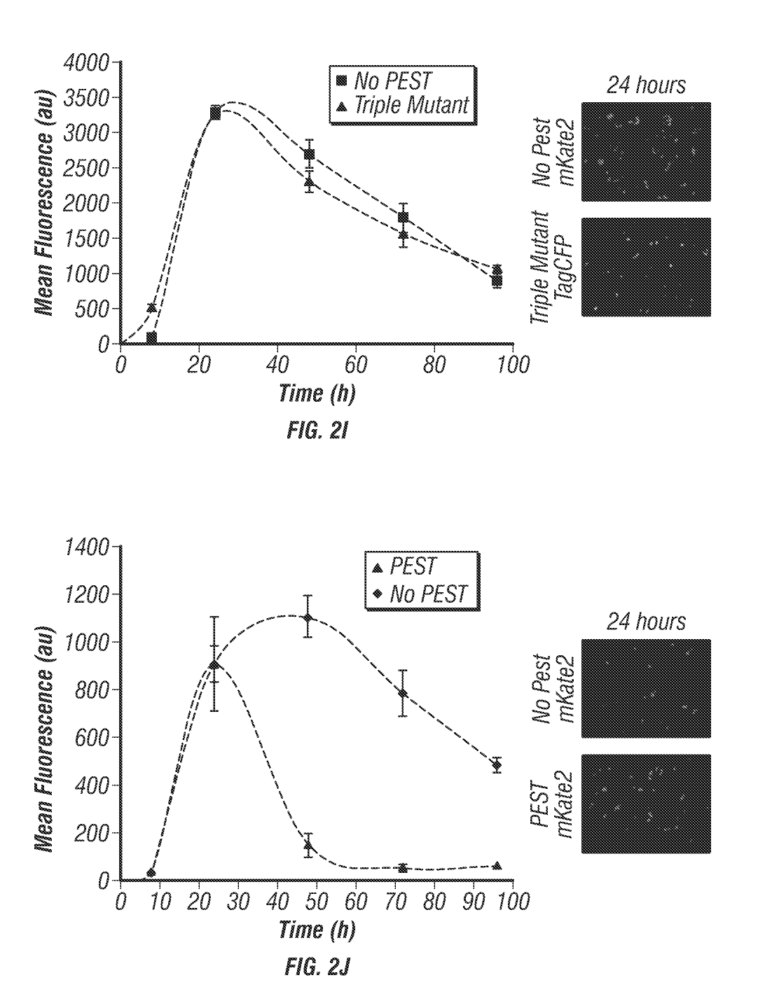

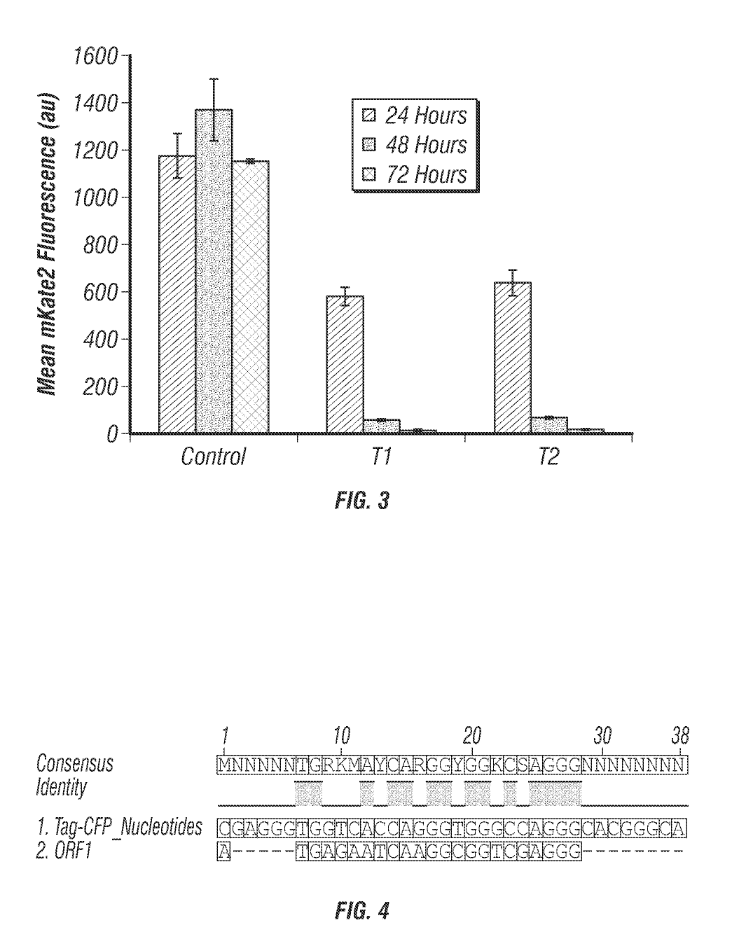

The BD LSRII FACS experiments were processed in FlowJo and elliptically gated for healthy cells based on the forward scatter side scatter recordings. The mean arbitrary fluorescence units of each protein were recorded for gated cells and error reported as standard deviation of replicate means. Background fluorescence was subtracted from each recording and calculated from empty control wells collected at each time point and averaged to generate a single background value. Each experiment utilizes a consistent voltage and processing workflow. Excitation occurred at 561 nm with a 545/35 band-pass (mKate2) and 445 nm with a 515/20 band-pass (Tag-CFP). Acquisition was performed with BD FACSDiva and a global polygon FSC/SSC gate was used to stop recording at a threshold of 50,000 gated events with all events saved and processed in FlowJo. Voltages were 280 for FSC, 290 for SSC, 385 for Tag-CFP and 490 for mKate2 with the exception of FIGS. 2A-B which used a voltage of 375 for mKate2.

Complete Pulse Plasmids with mKate2-PEST.

Guide RNA were constructed according to option B of the gRNA synthesis protocol from Mali et al². T1 gRNA used oligos P12 and P13, T2 gRNA used oligos P14 and P15 and the off-target gRNA used oligos P10 and P11. The gRNAs were sequenced using primer P9. Each gRNA construct was amplified along with its U6 promoter using primers P16 and P17 and cloned into Tag-CFP-N (Evrogen) using BsaI. To accomplish this BsaI cloning, each U6-gRNA amplicon was PCR purified and then digested with BsaI at 37° C.; the digested products were gel purified. Tag-CFP-N was digested with BsaI at 37° C., CIP treated and then gel purified. Each gRNA was ligated with the Tag-CFP-N backbone at a 1:3 ratio using T4 ligase at 4° C. overnight and then transformed into NEB-5-alpha high efficiency cells. The transformations were plated on Kanamycin plates and colonies were screened by test digestion with BsaI. These three vectors containing each U6-gRNA were subsequently used as a backbone for Cas9-2A-mKate2-PEST described in the following steps. To create Cas9-2A-mKate2-PEST, Cas9 was amplified from hCas9 (Addgene) using primers P1 and P2 and gel purified. Next, 2A-mKate2-PEST was amplified using primers P4 and P6 and gel purified. Both Cas9 and 2A-mKate2-PEST amplicons were combined using overlap extension PCR with primers P1 and P6. The completed amplicon from the overlap extension was then digested with NheI and NotI at 37° C. and gel purified. Each U6-gRNA-CMV-Tag-CFP backbone (T1, T2 and off-target) was digested with NheI and NotI at 37° C. gel purified. Next, the digested U6-gRNA backbones were ligated with the digested Cas9-2A-mKate2-PEST at a 1:1 ratio with T4 ligase overnight at 4° C. along with a negative reaction containing only the backbone. The following day, the ligations were transformed into XL10-Ultracompetent cells and spread on Kanamycin plates. Colonies were inoculated and screened by transfection. Sequencing was performed with primers P7 and P8.

Complete Pulse Plasmids with mKate2 No PEST.

U6-gRNAT1-CMV-Cas9-2A-mKate2-PEST was digested with ApaI at 25° C. for one hour and subsequently with FseI at 37° C. for one hour to remove mKate2-PEST and then gel purified. Next, mKate2 was amplified without PEST using primers P23 and P24 and digested with ApaI at 25° C. for one hour subsequently with FseI at 37° C. for one hour; the digestion was followed by gel purification. The two gel purified products were ligated 1:3 with T4 ligase overnight at 4° C. and transformed the following day in XL10-Ultracompetent cells. Sequencing was performed with primer P19 and verified by transfection.

Complete Pulse Plasmids with Targets Preceding Tag-CFP.

U6-gRNAT1-CMV-Cas9-2A-mKate2-PEST was digested with ApaI at 25° C. for one hour and subsequently with FseI at 37° C. for one hour to remove mKate2-PEST and then gel purified. Target T1 was added to the Tag-CFP-PEST sequence using primers P25 and P26. Single mutant target T1 was added to the Tag-CFP-PEST sequence using primers P27 and P26. Triple mutant target T1 was added to the Tag-CFP-PEST sequence using primers P28 and P26. Each of the three amplicons of Tag-CFP-PEST preceded by targets was digested with ApaI at 25° C. for one hour and subsequently with FseI at 37° C. for one hour; all three digestions were gel purified. Each Target-Tag-CFP-PEST was ligated with gel purified U6-gRNA-T1-CMV-Cas9-2A with T4 ligase overnight at 4° C. and transformed the following day in XL10-Ultracompetent cells.

Complete Pulse Plasmids with mKate2-PEST Driven by TRE3G.

The replication origin from U6-gRNAT1-CMV-Cas9-2A-mKate2-PEST was amplified using 34 and 35 while the TRE3G promoter was amplified using primers P32 and P33. The two amplicons were gel purified and then an overlap PCR was performed with primers P34 and P33. This product was digested with SpeI and NheI and then gel purified. U6-gRNAT1-CMV-Cas9-2A-mKate2-PEST was digested with SpeI and NheI, CIP treated, and then gel purified. The two gel purified digestions were joined with T4 ligase and transformed in XL10-Ultracompetent cells. The clones were screened for the correct orientation using primers P36 and P37 and then sequenced with primer P38. Subsequently, the construct was tested by transfection.

### Example 2

The inventors employ the well-established nuclease activity of Cas9, a Type II CRISPR associated protein, which has been codon optimized for expression in mammalian systems (Mali et al., 2013 and Pattanayak et al., 2013). As an RNA-guided system, Cas9 requires a gRNA to direct the binding of the nuclease to a 17-21 bp DNA protospacer target (Cong et al., 2013). Cas9 preferentially interrogates the protospacer adjacent motif (PAM) sequence (Sternberg et al., 2014) and upon encountering the gRNA target sequence it creates a double stranded break (DSB) in close proximity to the PAM (FIG. 1A).

The proposed strategy for precise inactivation and controllable gene delivery is based on a single delivery vehicle that packages all the necessary components for the CRISPR operation in addition to the desired gene product cassette. A top-level operation of the system as illustrated in FIG. 1B. Upon delivery, the Cas9 and gRNA modules are produced by their corresponding promoters together with the desired gene product (e.g., a fluorescent protein mKate2). The Cas9 and gRNA form a complex and interrogate potential targets. If the Cas9-gRNA complex recognizes a specific sequence in the open reading frame (ORF) of mKate2 it will cleave the vector and thus inhibit the protein production and trigger the vector degradation. In this case, the outcome in single-cell will be the disruption of the delivery vehicle and the gradual degradation of the products.

To explore the feasibility and properties of this intriguing mechanism, the inventors commenced experiments to test the efficiency of CRISPR/Cas9 in targeting the ORF of mKate2 using transient transfections in human kidney cells. Published results show that Cas9 has high nuclease activity and does allow for mismatches in the target sequence (Hsu et al., 2013 and Fu et al., 2013). In order to circumvent potential off-target effects, the inventors screened the mKate2 coding sequence targets against the human genome (Hsu et al., 2013). Subsequently, using Gibson assembly (Gibson et al., 2009), the inventors constructed the two gRNAs with the lowest off-target effect likelihood (Tables 1 and 2), and cloned them under the control of a U6 type III RNA polymerase promoter. As a negative control, the inventors constructed a third gRNA which has no targets in the mKate2 ORF (Table 1). Finally, the inventors cloned Cas9 and mKate2 under the control of a CMV promoter, separated by a self-cleaving Thosea asigna peptide sequence (2A-like). This peptide allows the expression of more than one gene product under the same promoter (Chang et al., 2009).

The inventors subsequently tested their gRNAs and Cas9-mKate2 expression system as two cotransfected plasmids, and validated the function of the Cas9-mKate2 coexpression system as well as the activity of the Cas9 with the engineered gRNAs (data not shown). The inventors then combined the gRNA, Cas9, mKate2 and their corresponding promoters into a single plasmid. This delivery vehicle utilizes a CMV promoter to expresses Cas9 and mKate2 separated by the 2A sequence and the U6 promoter for the gRNA (FIG. 1C). Considering the stability and corresponding long half-life of the fluorescent protein mKate2 (Kelmanson, 2009), the inventors introduced a PEST amino acid sequence into the C terminus of their fluorescent reporter mKate2 to catalyze its fast degradation (Li et al., 1998). The particular PEST domain (SwitchGear Genomics) was selected from Odc1, and has been shown to reduce the protein half-life to approximately two hours in mammalian cells (Li et al., 1998). The inventors finally verified that both single plasmids with gRNAs T1 and T2 degraded mKate2 while the off-target had no effect.

To characterize the behavior of the proposed delivery vehicle, the inventors transiently transfected human kidney embryonic cells (HEK293) and performed time-lapse microscopy starting five hours post-transfection. On population level (FIG. 1D), the inventors observed a pulse behavior in their CRISPR-based delivery vehicle (i.e., for the T1 gRNA) while the control (off-target gRNA) shows sustained mKate2 production. Single-cell analysis shows the mKate2 accumulation and degradation within a window of approximately 12 hours (FIG. 1E). Next, the inventors processed the raw microscopy files (Sage et al., 2010) and retrieved the mean fluorescence value for each time frame in single cells. Representative tracks of three cells further validated the pulse behavior and point to the peak fluorescence at approximately 7 hours after the mKate2 appearance (FIG. 1F). Interestingly, these representative tracks show a response with fast rise and slower degradation dynamics.

Prior to additional experimentation, to probe the dynamics of the response of the system, the inventors performed in silico experiments. The inventors used ordinary differential equations (ODE) to describe their CRISPR-based delivery vehicle (Tables 3 and 4) and utilized MATLAB to simulate the effects of parametric changes at the transcription, translation, and post-translational level.

The inventors' first observation is the ability to control the amplitude of the delivered product adjusting the vector mass (FIG. 1G). For the simulations, the inventors varied the amount of DNA copies. The model predicted that an increase in the DNA template would predominately increase the amplitude while having a mild effect on the residence time of the protein output (FIG. 1G). The inventors then investigated the effect of the output protein half-life to the response of the system. Specifically, the inventors performed simulations gradually decreasing the degradation rate of mKate2. As expected, lowering mKate2 degradation rate changed the trajectory of the pulse. Both the residence time and amplitude of the pulse increased as a result of greater protein stability (FIG. 1H). Finally, the inventors performed simulations gradually decreasing the affinity of the Cas9-gRNA complex for its target DNA (FIG. 1I). The simulations predict that lowering the Cas9-gRNA activity will again increase both the residence time and amplitude of the pulse. These computational results guided the inventors' subsequent experimentation. In particular, driven by these observations, the inventors engineered customized versions of the original system to probe the ability to control the residence time and amplitude of the delivered gene.

The inventors first removed the PEST domain from their system and performed a multipoint flow cytometry time-lapse. The population based results show that the delivery vehicle with the PEST fast-degradation tag yields a pulse of protein production with duration of approximately 48 hours (FIG. 2A). The inventors note that in difference to the single-cell microscopy-based tracking (i.e., FIG. 1E) the flow cytometry population measurements produce a histogram of fluorescent measurements from unsynchronized cells. While processing the microscopy data, the inventors observe that the cells start expressing the fluorescent signal at random times within the first 24 hours after the transfection (data not shown). Taken together, these observations explain the contrast between single and population based measurements.

The prediction is that a more stable mKate2 protein would lead to increased duration and residence time (FIG. 1H). Indeed, the removal of the PEST domain caused a marked increase in the duration of the pulse (for the same template concentration). The output signal remains present at 88 hours, yet at degradation phase (FIG. 2A). For both delivery vehicles, the inventors plot the mKate2 intensity versus the side scatter (SSC) (FIG. 2B). The system without PEST shows a significant population of cells with high signal at 24, 48 and 72 hours, with the number of cells and mean fluorescence intensity gradually decreasing (FIG. 2B). Representative microscopy snapshots (FIG. 2C) demonstrate the decrease in signal to near background in the mKate2-PEST fusion over the period from 24 to 48 hours post-transfection.

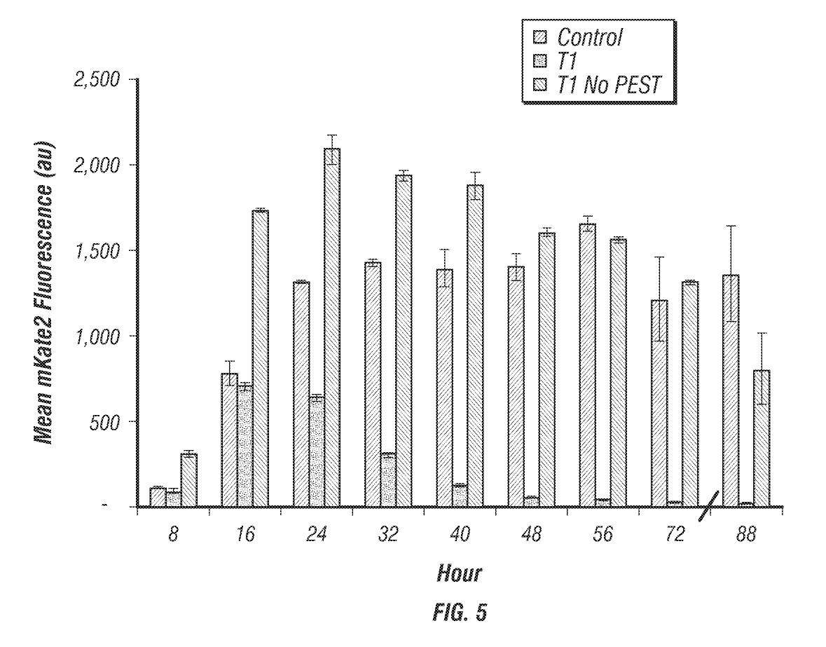

Subsequently, the inventors designed new mutant protospacers to reduce the affinity of Cas9-gRNA complex to the targeted DNA. To circumvent using the target within the ORF of the mKate2 gene, the inventors first replaced their reporter with Tag-CFP fused with the PEST domain. They screened the Tag-CFP ORF against the T1 mKate2 gRNA target and verified that there is no protospacer of sufficient similarity to trigger the Cas9-gRNA cleavage (FIG. 4) (Fu et al., 2013). The inventors proceeded to create three Tag-CFP versions of their vehicle delivery system with two mutant targets and the original T1 sequence immediately preceding the start codon of Tag-CFP (FIG. 2D). All three constructs contain the unmodified T1 gRNA expressed with the U6 promoter. The Cas9-gRNA complex crystal structure revealed that the PAM interacting domain interrogates DNA separately from the region that presents the gRNA to the DNA (Nishimasu et al., 2014). In their case, the transcripts for gRNAs do not contain the PAM sequence and the Cas9-gRNA complex should tolerate any nucleotide in the N position of the PAM sequence (Sternberg et al., 2014). These results are indeed aligned with these findings, indicating a mutation at the N position (G to T) yields similar pulse behavior to the original T1 target control (FIG. 2E). The triple mutant contains the single mutation with an additional two mutated nucleotides proximal the PAM (FIG. 2D). This mutant exhibited increased residence time and amplitude versus the original target control experiment (FIG. 2E). The results of FIG. 2e are also presented as raw flow cytometry data of the Tag-CFP intensity versus SSC, and show the population of cells at different time points (FIG. 5). Representative microscopy snapshots demonstrate the pulse behavior of the original target and single mutant while the triple mutant maintains high fluorescence at 48 hours (FIG. 2F).

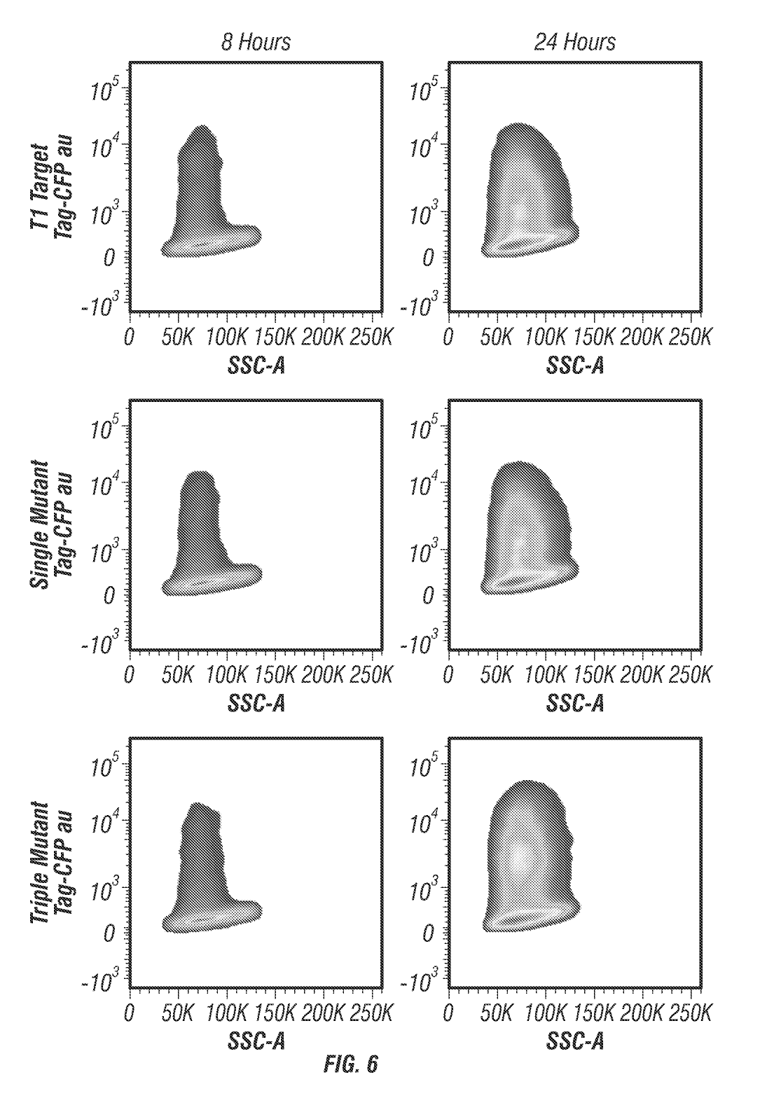

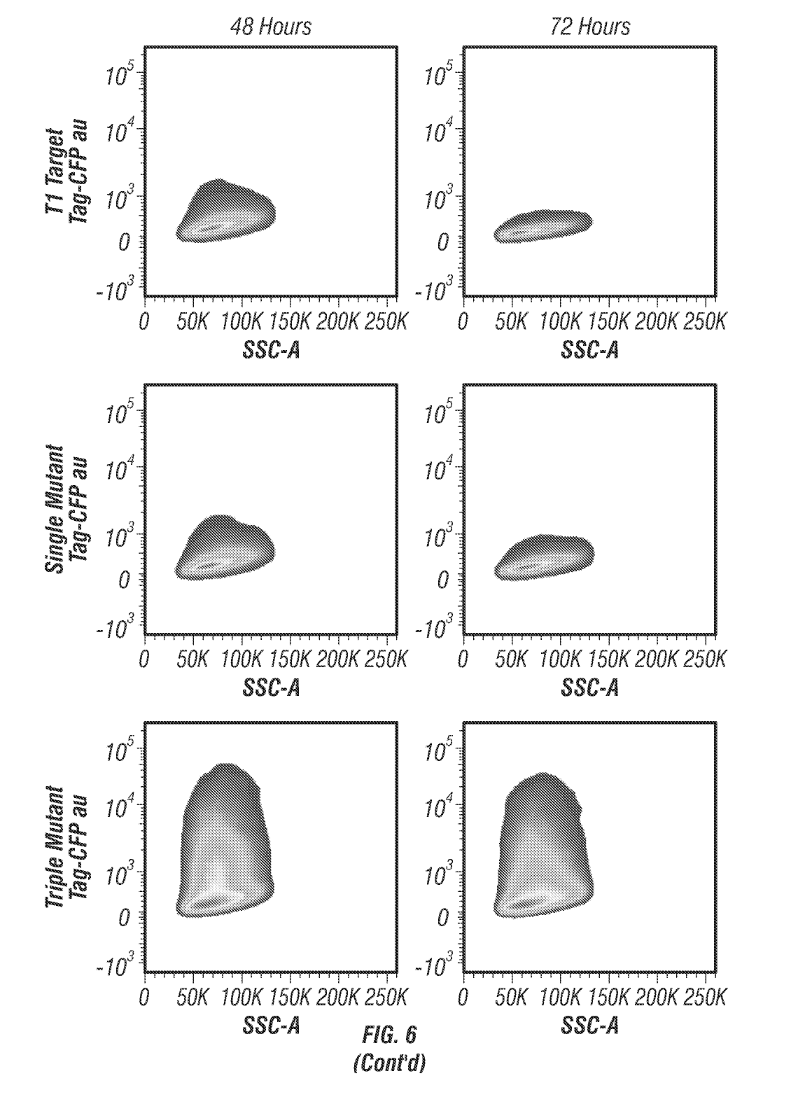

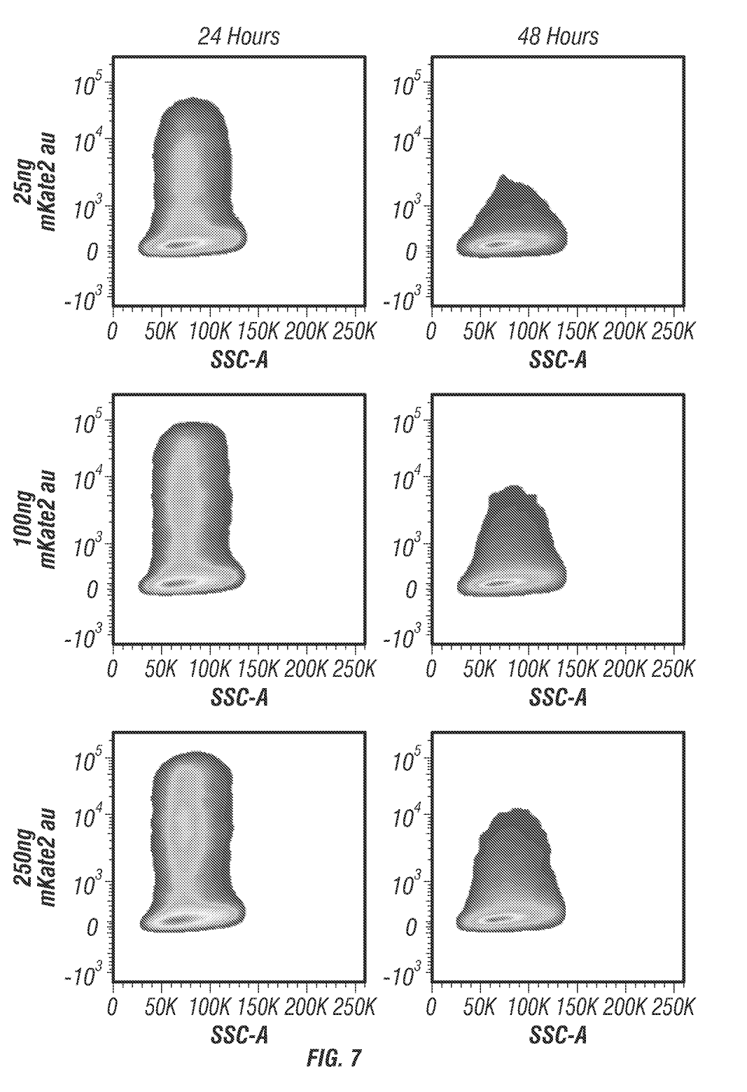

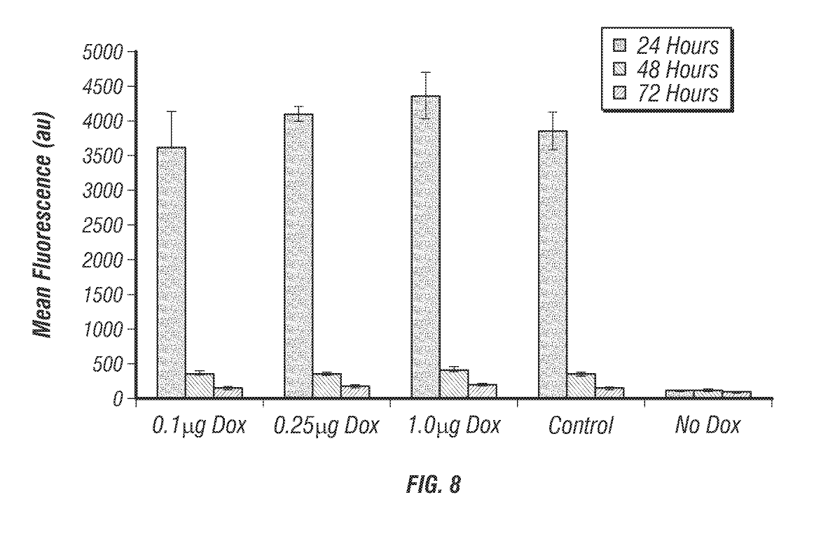

Furthermore, based on their simulation results (FIG. 1G), the inventors directed their attention on controlling the output amplitude. These simulations show that the plasmid mass can be adjusted to control the product amplitude. Accordingly, the inventors performed transfections at 25 ng, 100 ng and 250 ng. The inventors observed increasing intensity over the higher transfection amounts at 24 hours post-transfection (FIG. 2G). Additionally, the average intensity values of mKate2, as recorded by flow cytometry, returned to near background levels for all the concentrations at 48 hours (FIG. 2H, FIG. 6). Here, the ability to control the amplitude while maintaining similar residence time provides an effective means for controlling protein delivery dosage. The inventors were also interested to probe their system using an inducible promoter driving the production of the Cas9 and mKate2 transcript. To accomplish this, the inventors replaced their CMV promoter with the TRE3G system (Clontech) and tested various levels of Doxycycline (DOX) in Tet-on cells (HEK293 cells that harbor a copy of the transcription factor rtTA stably integrated in the genome). The inventors observed that they can indeed control the amplitude of the output protein and maintain the same residence time (FIG. 2I), yet the high sensitivity of the TRE3G promoter to the rtTA-DOX complex allowed a narrow output amplitude range (FIG. 2I). The control experiments are included in FIGS. 7 and 8.

Considering the capacity to fine-tune the amplitude of this delivered protein, the last objective was to compare the dynamics of the three constructs by adjusting the transfection mass in order to achieve the same maximum amplitude. The inventors discovered that the 10:1 mass ratio between the triple mutant and the system without the PEST domain yields the same maximum output levels (FIG. 2J). Interestingly, lowering the activity of the Cas9-gRNA complex by mutating the targets yielded a similar pulse trajectory to that of the mKate2 lacking a PEST domain (FIG. 2J). Finally, the inventors transfected the original plasmid and the vector lacking the PEST domain, and again the inventors show that they can achieve the same maximum output yet control the residence time of delivery (FIG. 2K). Specifically, the output lacking the PEST domain remains stable in the cellular milieu approximately twice as long as the output carrying the fast degradation domain.

Here, the inventors present a “self-destructing message,” in the form of plasmid DNA that can only be read a finite number of times by the cell. As the messenger RNAs are transcribed and then translated the protein Cas9 in complex with the gRNA returns to the plasmid and creates double stranded breaks, effectively disrupting its function and destroying the delivery mechanism.

The proposed methodology complements current tools (Deans et al., 2007) to produce genetic switches in synthetic architectures. The system can guarantee the elimination of leakage state in a regulated gene, albeit at a non-reversible function. Moreover, the inventors show that specific parameters of the system can be used to fine-tune the response of the output protein dynamics. CRISPR Cas9 systems have the potential to build complex gene regulatory modules (Kiani et al., 2014) and they envision future applications where the inventors' delivery vehicle is used in synthetic architectures and for controlled therapeutic gene delivery.

Lastly, the proposed mechanism can be adopted to produce trace-free delivery for potential applications in protection of genetic material intellectual property. Introducing multiple, strategically located targets in the delivery vehicle can result to short fragments and accelerate the degradation process by endonucleases in addition to randomizing the original code, rendering it unrecoverable.

TABLE 1
gRNAs

| | | SEQ ID |
|---|---|---|
| gRNA Name | gRNA Sequence | NO: |
| T1: mKate2 Open | ATGAGAATCAAGGCGGTCGA | 1 |
| Reading Frame | | |
| T2: mKate2 Open | CATGAGAATCAAGGCGGTCG | 2 |
| Reading Frame | | |
| Off-Target: No match | TAGGCAAGAGTGCCTTGACG | 3 |
| in complete plasmids | | |

TABLE 2
Screened gRNA targets for the mKate2 open reading
frame*

| | | | | SEQ ID |
|---|---|---|---|---|
| # | gRNA | PAM | Score | NO: |
| 1 (T1) | ATGAGAATCAAGGCGGTCGA | GGG | 91 | 4 |
| 2 (T2) | CATGAGAATCAAGGCGGTCG | AGG | 90 | 5 |
| 3 | GGCGAAGGCAAGCCCTACGA | GGG | 88 | 6 |
| 4 | AAGTGCACATCCGAGGGCGA | AGG | 88 | 7 |
| 5 | CACTTCAAGTGCACATCCGA | GGG | 83 | 8 |
| 6 | AGAATCAAGGCGGTCGAGGG | CGG | 83 | 9 |
| 7 | ATGGTCTGGGTGCCCTCGTA | GGG | 82 | 10 |
| 8 | GGGCGAAGGCAAGCCCTACG | AGG | 82 | 11 |
| 9 | GTAGGGCTTGCCTTCGCCCT | CGG | 82 | 12 |
| 10 | CCACTTCAAGTGCACATCCG | AGG | 80 | 13 |
| 11 | CATGGTCTGGGTGCCCTCGT | AGG | 77 | 14 |
| 12 | GCCAGGATGTCGAAGGCCAA | GGG | 77 | 15 |
| 13 | CTCGACCGCTTGATTCTCA | TGG | 75 | 16 |
| 14 | AGCCAGGATGTCGAAGGCGA | AGG | 73 | 17 |
| 15 | TTTGCTGCCGTACATGAAGC | TGG | 73 | 18 |

*-Only the targets with the highest quality score are shown.

TABLE 3
Ordinary Differential Equation

[Pulse Plasmid] -> mRNACas9_2A_mKate2 + [Pulse Plasmid]
mRNACas9_2A_mKate2 -> null
mRNACas9_2A_mKate2 -> mRNACas9_2A_mKate2 + Cas9
Cas9 -> null
mRNACas9_2A_mKate2 -> mRNACas9_2A_mKate2 + mKate2
mKate2 -> null
[Pulse Plasmid] -> gRNA + [Pulse Plasmid]
gRNA -> null
Cas9 + gRNA -> Cas9-gRNA
Cas9-gRNA -> null
[Pulse Plasmid] + Cas9-gRNA -> [Pulse Plasmid-Cas9-gRNA]
[Pulse Plasmid-Cas9-gRNA] -> null

TABLE 4

| Parameter | Value (1/s)¹ |
|---|---|
| k_mRNA_Cas9_2A_mKate2 | 0.08227 |
| k_mRNA_Cas9_mKate2_deg | 0.004214 |
| k_gRNA | 0.00004214 |
| k_gRNA_deg | 0.004214 |
| k_pr_Cas9 | 7.59E−06 |
| k_pr_Cas9_deg | 9.20E−07 |
| k_pr_mKate2 | 7.30E−05 |
| k_pr_mKate2_deg | 2.77E−06 |
| k_Cas9_gRNA_Complex | 1.00E−06 |
| k_Cas9_gRNA_Complex_deg | 0.004214 |
| k_Cas9_gRNA_Plasmid_cutting | 1.00E−03 |

TABLE 5
Oligos

| Identifier | Description | Oligo | SEQ ID NO: |
|---|---|---|---|
| P1 | Cas9 Forward with | GCCACCGCTAGCCTTGCCACCATGGA | 19 |
| | NheI | CAAGAAGTACTCC | |
| P2 | Cas9 Reverse 2A | AAGACTTCCTCTGCCCTCAGCCACCTT | 20 |
| | Overlap | CCTCTTCTTCTTGGGGTCAGC | |
| P4 | 2A Peptide FWD (for | GCTGAGGGCAGAGGAAGTCTTC | 21 |
| | mKate2) no Target | | |
| P6 | PEST Reverse with | gccaccGCGGCCGCTTAGACGTTGATC | 22 |
| | TAA (for mKate2) | CTGGCGCTG | |
| | with NotI (preserves | | |
| | Poly A tail of CFP | | |
| | backbone) | | |
| P7 | mKate2 Forward | ACCGAGACCCTGTACCCCGC | 23 |
| P8 | mKate2 Reverse | CAGGGCCATGTCGGCTCTGC | 24 |
| P9 | U6 gRNA seq | GGAACCAATTCAGTCGACTGGATC | 25 |
| | Forward | | |
| P10 | Off Target gRNA | TTTCTTGGCTTTATATATCTTGTGGAA | 26 |
| | Forward | AGGACGAAACACCGAGGCAAGAGTG | |
| | | CCTTGACG | |
| P11 | Off Target gRNA | GACTAGCTTATTTTAACTTGCTATTT | 27 |
| | Reverse | CTAGCTCTAAAACCGTCAAGGCACTC | |
| | | TTGCCTC | |
| P12 | Screened Target 1 | TTTCTTGGCTTTATATATCTTGTGGAA | 28 |
| | mKate2 CDS Forward | AGGACGAAACACCGTGAGAATCAAG | |
| | | GCGGTCGA | |
| P13 | Screened Target 1 | GACTAGCCTTATTTTAACTTGCTATTT | 29 |
| | mKate2 CDS Reverse | CTAGCTCTAAAACTCGACCGCCTTGAT | |
| | | TCTCAC | |
| P14 | Screened Target 2 | TTTCTTGGCTTTATATATCTTGTGGAA | 30 |
| | mKate2 CDS Forward | AGGACGAAACACCGATGAGAATCAA | |
| | | GGCGGTCG | |
| P15 | Screened Target 2 | GACTAGCTTATTTTAACTTGCTATTT | 31 |
| | mKate2 CDS Reverse | CTAGCTCTAAAACCGACCGCCTTGATT | |
| | | CATC | |
| P16 | gRNA Extract | GCCACCccaccgagaccTCAGTCGACTG | 32 |
| | Forward BsaI (BsaI | GATCCGGTA | |
| | native to Evrogen | | |
| | CFP) | | |
| P17 | gRNA Extract Reverse | GCCACCggtctcggtggGATCCACTAGTA | 33 |
| | BsaI | ACGGCCG | |
| P23 | ApaI mKate2 FWD | gccaccGGGCCCATGGTGAGCGAGCTG | 34 |
| | | ATTAAGGAGAA | |
| P24 | FseI mKate2 REV with | gccaccGGCCGGCTTATCTGTGCCCCA | 35 |
| | STOP | GTTTGCTAGG | |
| P25 | CFP PEST No | gccaccGGGCCCATGAGAATCAAGGCG | 36 |
| | Mutation F (open | GTCGAGGGgATGAGCGGGGGCGAGG | |
| | reading frame #1 | AGCTGTTC | |
| | from mKate2) | | |
| P26 | CFP PEST REV | gccaccGGCCGGCCTTAGACGTTGATC | 37 |
| | | CTGGCGCTGGC | |
| P27 | CFP PEST 1 Mutation | gccaccGGGCCCATGAGAATCAAGGCG | 38 |
| | F (G-T,A-C,C-A,T-G) | GTCGATGGgATGAGCGGGGGCGAGG | |
| | | AGCTGTTC | |
| P28 | CFP PEST 3 Mutation | gccaccGGGCCCATGAGRATCAAGGCT | 39 |
| | F (G-T,A-C,C-A,T-G) | GTAGATGGgATGAGCGGGGGCGAGG | |
| | | AGCTGTTC | |
| P32 | TRE-3G-FWD | TGTGGATAACCGTATTACCGCCCCGT | 40 |
| | (truncate left cmv | ACACGCCACCTCGACATA | |
| | min) with rep origin | | |
| | overlap | | |
| P33 | TRE-3G REV with | gccaccGCTAGCCGGAAAGTTGGTATA | 41 |
| | NheI | AGACAAAAGTGTTGTGG | |
| P34 | Plasmid Rep Origin | CGGCCGTTACTAGTGGATCCGC | 42 |
| | FWD with SpeI | | |
| P35 | Plasmid | GGCGGTAATACGGTTATCCACAGAAT | 43 |
| | Rep Origin | CAGG | |
| | REV | | |
| P36 | CPCR 3G FWD | TGGAAAGGACGAAACACCGT | 44 |
| | Anneal 52 | | |
| P37 | CPCR 3G REV- | TTTTCTACGGGGTCTGACGC | 45 |
| | Product is 473 | | |
| P38 | Cas9 REV (near | TTTGCTCGGCACCTTGTACT | 46 |
| | beginning) | | |

Controllable gene delivery via vector-based systems remains a formidable challenge in mammalian synthetic biology and a desirable asset in gene therapy applications. Viral and non-viral delivery techniques result in prolonged transient expression of transgenes, extreme fluctuations in stoichiometry between different products in individual cells, and often undesirable functional genomic integrations.

The inventors employ the well-established nuclease activity of Cas9, a Type II clustered regularly interspaced short palindromic repeats (CRISPR) associated protein, which has been codon-optimized for expression in mammalian systems. As an RNA-guided system, Cas9 requires a gRNA (guide RNA) to direct the binding of the nuclease to a 17-21 bp DNA protospacer target. Cas9 preferentially binds to a protospacer adjacent motif (PAM) and upon encountering the gRNA target sequence it creates a double stranded break (DSB) in close proximity to the PAM.

The inventors introduce a new mechanism, a self-regulated “message” in the form of plasmid DNA that can only be read a finite number of times by the cell. As the messenger RNAs are transcribed and then translated, the protein Cas9 in complex with the gRNA binds the delivery cassette, disrupting the delivery mechanism permanently (for double-stranded breaks) or reversibly (for nicks).

Using experiments in human embryonic kidney cells, the inventors show that they can fine-tune the response of the output protein dynamics. The ability to control the amplitude and residence time provides an effective means for controlling protein delivery dosage which will have disruptive impact for a wide range of synthetic biology and gene therapy applications.

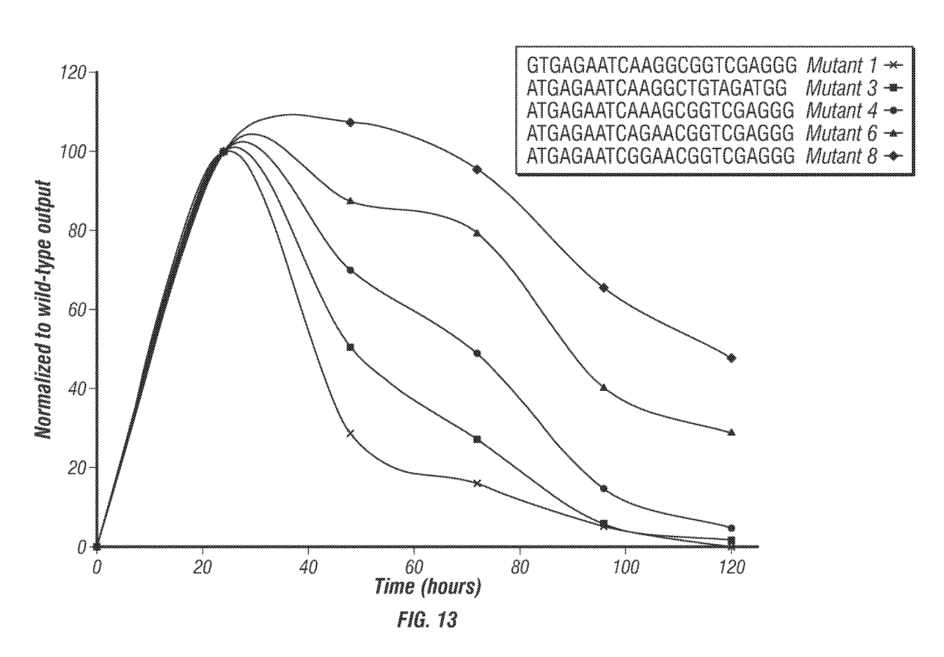

The inventors note that the proposed self-cleaving mechanism can be adopted to produce trace-free delivery for potential applications in protection of genetic material intellectual property. Introducing multiple, strategically located targets in the delivery vehicle can result in short fragments that randomize the original code, rendering it unrecoverable (FIG. 13).

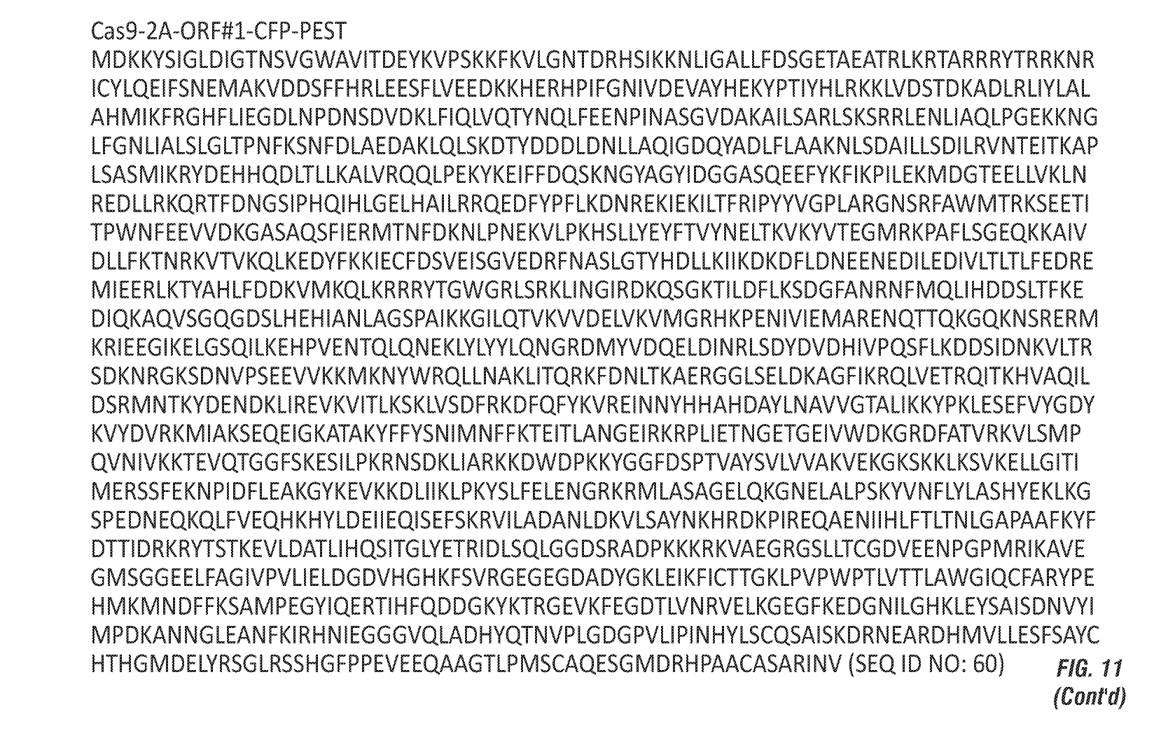

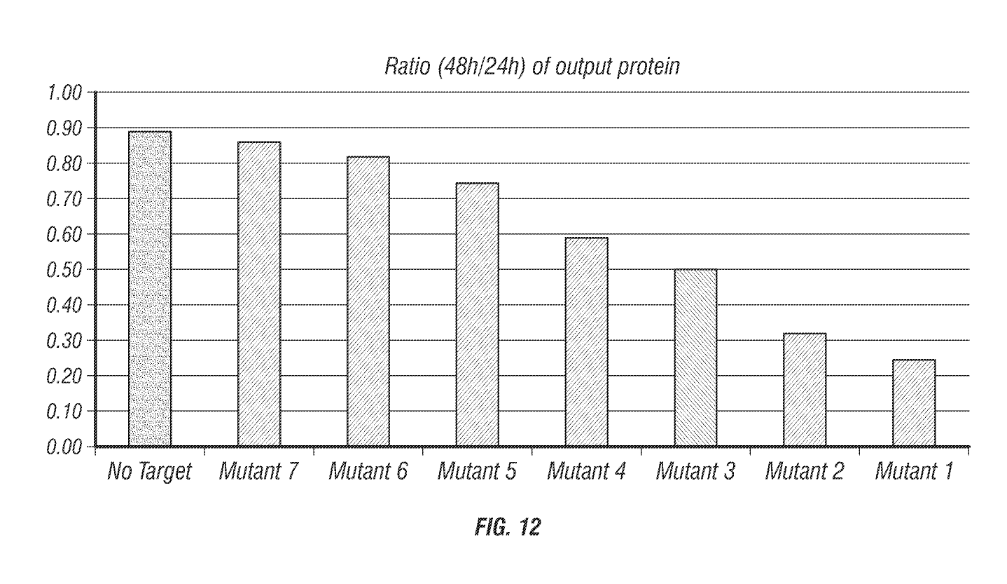

The inventors modified the gRNA target of the T1 gRNA and designed several new mutants. FIG. 12 contains the quantified measurements of six new mutants, the triple mutant (mutant 3) and a control (no target). As an example, the system that has no targets is approximately stable between 24 and 48 hour measurements (population-based flow cytometry). In contrast, mutant 3 (from NAR) at 48 hours is approximately 50% of the 24 hour levels. As illustrated FIG. 13, the inventors produced a spectrum of responses for the residence time of the delivered fluorescent protein. The specific target sequences are illustrated in the Table 6:

TABLE 6
Target Sequences

| Label | Target Sequence | SEQ ID NO: |
|---|---|---|
| No target | No Target | |
| Mutant 7 | ATGAGAATCGGAATGGTCGAGGG | 47 |
| Mutant 8 | ATGAGAATCGGAACGGTCGAGGG | 48 |
| Mutant 6 | ATGAGAATCAGAACGGTCGAGGG | 49 |
| Mutant 5 | ATGAGAATCAGAGCGGTCGAGGG | 50 |
| Mutant 4 | ATGAGAATCAAAGCGGTCGAGGG | 51 |
| Mutant 3 | ATGAGAATCAAGGCTGTAGATGG | 52 |
| (manuscript) | | |
| Mutant 2 | GCGAGAATCAAGGCGGTCGAGGG | 53 |
| Mutant 1 | GCTGAGAATCAAGGCGGTCGAGGG | 54 |
| Wild-type | ATGAGAATCAAGGCGGTCGAGGG | 4 |
| (manuscript) | | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
Angel et al., Cell, 49:729, 1987b.
Angel et al., Mol. Cell. Biol., 7:2256, 1987a.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Benvenisty and Neshif, Proc. Natl. Acad. Sci. USA, 83:9551-9555, 1986.
Berkhout et al., Cell, 59:273-282, 1989.
Bhaysar et al., Genomics, 35(1):11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Coffin, In: Virology, Fields et al. (Eds.), Raven Press, NY, 1437-1500, 1990.
Cohen et al., J. Cell. Physiol., 5:75, 1987.
Cong et al., Sciencexpress, 2013.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deans et al., Cell., 130:363-372, 2007.
Deschamps et al., Science, 230:1174-1177, 1985.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Foecking and Hofstetter, Gene, 45(1):101-105, 1986.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fu et al., Nat. Biotechnol., 2013.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gibson et al., Nature methods. 6:343-345, 2009.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Horlick and Benfield, Mol. Cell. Biol., 9:2396, 1989.
Horwich et al., J. Virol., 64:642-650, 1990.
Hsu et al., Nat. Biotechnol., 2013.
Hsu et al., Nall Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kelmanson, Nature Methods. 6, 2009.
Kiani et al., Nat Meth. advance online publication, 2014.
Kiledjian et al., Ma Cell. Biol., 8:145, 1988.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Li et al., J. Biol. Chem., 273:34970-34975, 1998.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Nat. Biotechnol. 31:833-838, 2013.
Mali et al., Science 339 (6121):823-826, 2013a.
Mann et al., Cell, 33:153-159, 1983.
Markowitz et al., J. Virol., 62:1120-1124, 1988.
McNeall et al., Gene, 76:81, 1989.
Miksicek et al., Cell, 46:203, 1986.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nishimasu et al., Cell., 156:935-949, 2014.
Ondek et al., EMBO J., 6:1017, 1987.
Ornitz et al., Mol. Cell. Biol., 7:3466, 1987.
Palmiter et al., Cell, 29:701, 1982.
Palmiter et al., Nature, 300:611, 1982.
Paskind et al., Virology, 67:242-248, 1975.
Pattanayak et al., Nat. Biotechnol., 2013.
Pech et al., Mol. Cell. Biol., 9:396, 1989.
Perales et al., Proc. Natl. Acad. Sci. USA, 91(9):4086-4090, 1994.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sage et al., Cell. Div., 5:17-1028-5-17, 2010.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Sternberg et al., Nature, 2014.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA, 87(9):3410-3414, 1990.
Wang and Calame, Cell, 47:241, 1986.
Weber et al., Cell, 36:983, 1984.
Weinberger et al. Mol. Cell. Biol., 8:988, 1984.
Winoto and Baltimore, Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J Biol. Chem., 262:4429-4432, 1987.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yutzey et ah Mol. Cell. Biol., 9:1397, 1989.
Zelenin et al., FEBS Lett., 280:94-96, 1991.
Ziober and Kramer, J. Bio. Chem., 271(37):22915-22922, 1996.

1. A vector comprising:
(a) a heterologous gene sequence under the control of a promoter active in eukaryotic cells;
(b) a coding region for Cas9 protein under the control of a promoter active in eukaryotic cells;
(c) a coding region for a gRNA under the control of a promoter active in eukaryotic cells wherein said gRNA targets a sequence in said delivery vector encoding said gene of interest; and
(d) sequences required for expression of said heterologous gene sequence, Cas9 coding region and said gRNA coding region.

2. The vector of claim 1, wherein said vector is a non-viral vector.

3. The vector of claim 1, wherein said vector is a viral vector.

4. The vector of claim 3, wherein said viral vector is a retroviral vector, a herpesviral vector, an adenoviral vector, an adeno-associated viral vector or a poxviral vector.

5. The vector of claim 1, wherein said heterologous gene sequence encodes a therapeutic gene product or a non-therapeutic detectable and/or selectable gene product.

6. (canceled)

7. The vector of claim 1, wherein said promoters are active in mammalian cells.

8. (canceled)

9. The vector of claim 1, wherein one promoter directs expression of both said heterologous gene sequence and said Cas9 coding region.

10. The vector of claim 1, wherein said heterologous gene sequence further comprises a coding sequence for a protein degradation tag.

11. A method of expressing a protein of interest in a target cell comprising
(a) transferring a vector into said cell, said vector comprising:
(i) a gene sequence encoding said protein of interest under the control of a promoter active in eukaryotic cells;
(ii) a coding region for Cas9 protein under the control of a promoter active in eukaryotic cells;
(iii) a coding region for a gRNA under the control of a promoter active in eukaryotic cells wherein said gRNA targets a sequence in said delivery vector encoding said gene of interest; and
(iv) sequences required for expression of said gene sequence encoding said protein of interest, Cas9 coding region and said gRNA coding region, and
(b) culturing said cell under conditions supporting expression of said protein of interest.

12. The method of claim 11, wherein said viral vector is a retroviral vector, a herpesviral vector, an adenoviral vector, an adeno-associated viral vector or a poxviral vector.

13. The method of claim 11, wherein said protein of interest is a therapeutic gene product or a non-therapeutic detectable and/or selectable gene product.

14. (canceled)

15. The method of claim 11, wherein said promoters are active in mammalian cells.

16. (canceled)

17. The method of claim 11, wherein one promoter directs expression of both said gene sequence encoding said protein of interest and said Cas9 coding region.

18. The method of claim 11, wherein said gene sequence encoding said protein of interest further comprises a coding sequence for a protein degradation tag.

19. The method of claim 11, wherein said Cas9 coding region further comprises a coding sequence for a protein degradation tag.

20. The method of claim 11, wherein said cell is an animal cell or a plant cell.

21-22. (canceled)

23. The method of claim 11, wherein said protein of interest is endogenous to said cell.

24. The method of claim 11, wherein said protein of interest is not endogenous to said cell.

25. The method of claim 23, wherein said protein of interest is underexpressed or not expressed in said cell as relative to the normal level in a comparable cell.

26. The method of claim 11, wherein said cell is a cancer cell or a neuronal cell.
